SEQUENCE LISTING (44 sequences)

SEQ ID NO: 1 - protein, 12 aa, Artificial Sequence
  synthetic polypeptide
PQVTRGDVFTMP

SEQ ID NO: 2 - protein, 14 aa, Artificial Sequence
  Synthetic polypeptide
KGGPQVTRGDVTMP

SEQ ID NO: 3 - protein, 7 aa, Artificial Sequence
  Synthetic polypeptide
GRGDSPK

SEQ ID NO: 4 - protein, 15 aa, Artificial Sequence
  Synthetic polypeptide
KGGAVTGRGDSPASS

SEQ ID NO: 5 - protein, 15 aa, Artificial Sequence
  Synthetic polypeptide
KGGNGEPRGDTYRAY

SEQ ID NO: 6 - protein, 9 aa, Artificial Sequence
  Synthetic polypeptide
KGGGFRGDQ

SEQ ID NO: 7 - protein, 16 aa, Artificial Sequence
  Synthetic polypeptide
KGGCKRARGDDMDDYC

SEQ ID NO: 8 - protein, 13 aa, Artificial Sequence
  Synthetic polypeptide
KGGCGGFHRRIKA

SEQ ID NO: 9 - protein, 14 aa, Artificial Sequence
  Synthetic polypeptide
KGGGWKTSRTSHTC

SEQ ID NO: 10 - protein, 15 aa, Artificial Sequence
  Synthetic polypeptide
KGGPQVTRGDVFTMP

SEQ ID NO: 11 - protein, 9 aa, Artificial Sequence
  Synthetic polypeptide
KGGGRGDTP

SEQ ID NO: 12 - protein, 14 aa, Artificial Sequence
  Synthetic polypeptide
KGGNGEPRGDTRAY

SEQ ID NO: 13 - protein, 20 aa, Artificial Sequence
  Synthetic polypeptide
KGGGQKCIVQTTSWSQCSKS

SEQ ID NO: 14 - protein, 13 aa, Artificial Sequence
  Synthetic polypeptide
KYGLALERKDHSG

SEQ ID NO: 15 - protein, 23 aa, Artificial Sequence
  Synthetic polypeptide
KGGSINNNRWHSIYITRFGNMGS

SEQ ID NO: 16 - protein, 15 aa, Artificial Sequence
  Synthetic polypeptide
KGGTWYKIAFQRNRK

SEQ ID NO: 17 - protein, 15 aa, Artificial Sequence
  Synthetic polypeptide
KGGTSIKIRGTYSER

SEQ ID NO: 18 - protein, 16 aa, Artificial Sequence
  Synthetic polypeptide
KYGTDIRVTLNRLNTF

SEQ ID NO: 19 - protein, 16 aa, Artificial Sequence
  Synthetic polypeptide
KYGSETTVKYIFRLHE

SEQ ID NO: 20 - protein, 15 aa, Artificial Sequence
  Synthetic polypeptide
KYGKAFDITYVRLKF

SEQ ID NO: 21 - protein, 15 aa, Artificial Sequence
  Synthetic polypeptide
KYGAASIKVAVSADR

SEQ ID NO: 22 - protein, 15 aa, Artificial Sequence
  Synthetic polypeptide
KYGRKRLQVQLSIRT

SEQ ID NO: 23 - protein, 13 aa, Artificial Sequence
  Synthetic polypeptide
KGGRNIAEIIKDI

SEQ ID NO: 24 - protein, 11 aa, Artificial Sequence
  Synthetic polypeptide
KGGCGGDGEAG

SEQ ID NO: 25 - protein, 14 aa, Artificial Sequence
  Synthetic polypeptide
KGGCWKTSLTSHTC

SEQ ID NO: 26 - protein, 12 aa, Artificial Sequence
  synthetic polypeptide
KGGGASGERGPX

SEQ ID NO: 27 - protein, 12 aa, Artificial Sequence
  synthetic polypeptide
KGGGLXGERGRX

SEQ ID NO: 28 - protein, 12 aa, Artificial Sequence
  Synthetic polypeptide
KGGGFXGERGVQ

SEQ ID NO: 29 - protein, 15 aa, Artificial Sequence
  synthetic polypeptide
TAGSCLRKFSTMGGK

SEQ ID NO: 30 - protein, 15 aa, Artificial Sequence
  synthetic polypeptide
KGGGPXGFXGERGPX

SEQ ID NO: 31 - protein, 12 aa, Artificial Sequence
  synthetic polypeptide
NGEPRGDTYRAY

SEQ ID NO: 32 - protein, 12 aa, Artificial Sequence
  synthetic polypeptide
AVTGRGDSPASS

SEQ ID NO: 33 - protein, 10 aa, Artificial Sequence
  synthetic polypeptide
RNIAEIIKDI

SEQ ID NO: 34 - protein, 11 aa, Artificial Sequence
  synthetic polypeptide
NGEPRGDTRAY

SEQ ID NO: 35 - protein, 12 aa, Artificial Sequence
  synthetic polypeptide
TSIKIRGTYSER

SEQ ID NO: 36 - protein, 12 aa, Artificial Sequence
  synthetic polypeptide
TWYKIAFQRNRK

SEQ ID NO: 37 - protein, 20 aa, Artificial Sequence
  synthetic polypeptide
SINNNRWHSIYITRFGNMGS

SEQ ID NO: 38 - protein, 17 aa, Artificial Sequence
  synthetic polypeptide
GQKCIVQTTSWSQCSKS

SEQ ID NO: 39 - protein, 17 aa, Artificial Sequence
  synthetic polypeptide
KGGKDGEPRGDTYRATD

SEQ ID NO: 40 - protein, 13 aa, Artificial Sequence
  synthetic polypeptide
KGGLEPRGDTYRD

SEQ ID NO: 41 - protein, 17 aa, Artificial Sequence
  syntehtic polypeptide
KGGCNGEPRGDTYRATC

SEQ ID NO: 42 - protein, 13 aa, Artificial Sequence
  synthetic polypeptide
KGGCEPRGDTYRC

SEQ ID NO: 43 - protein, 15 aa, Artificial Sequence
  synthetic polypeptide
KGGAVTGDGNSPASS

SEQ ID NO: 44 - protein, 10 aa, Artificial Sequence
  synthetic polypeptide
KGGGFRGDGQ

(19) United States
(12) Patent Application Publication
(10) Pub. No.: US 2012/0052580 A1
(43) Pub. Date: Mar. 1, 2012

(54) PEPTIDE-MODIFIED SURFACES FOR CELL CULTURE

(76) Inventors: Sadashiva Karnire Pai, Painted Post, NY (US); Odessa Natalie Petzold, Elmira, NY (US); Simon Kelly Shannon, Horseheads, NY (US); David Michael Weber, Big Flats, NY (US); Theresa Chang, Painted Post, NY (US); Jin Liu, Painted Post, NY (US)

(21) Appl. No.: 13/216,621

(22) Filed: Aug. 24, 2011

Related U.S. Application Data
(60) Provisional application No. 61/377,715, filed on Aug. 27, 2010.

(51) Int. Cl. C12N 5/071; C12N 5/0797; C12N 5/0735; C12N 5/0793; C12N 5/079

(52) U.S. Cl. 435/396

(57) ABSTRACT

A cell culture article including a pre-blocked, peptide-modified, polymer surface of the formula (I), where AAj represents at least one covalently bonded peptide, j is an integer of from 5 to 50, m, n, o, Sur, X, R, R′, and the mer ratio (m-o:n:o), including salts thereof, are as defined herein. Methods for making and using the cell culture article, as defined herein, are also disclosed.

### CROSS-REFERENCE TO RELATED APPLICATION(S)

This application claims the benefit of priority under 35 U.S.C. §119 of U.S. Provisional Application Ser. No. 61/377,715, filed on Aug. 27, 2010, the content of which is relied upon and incorporated herein by reference in its entirety.

### BACKGROUND

The disclosure generally relates to synthetically-modified polymer surfaces for cell culture applications.

### SUMMARY

The disclosure provides biologically-compatible, synthetically-modified polymer surfaces and articles for cell culture applications, and methods for making and using the cell culture articles.

### BRIEF DESCRIPTION OF THE DRAWING(S)

In embodiments of the disclosure:

FIG. 1 schematically shows a process used to prepare cell culture surfaces including modifying a reactive polymer surface with biologically-compatible peptide sequences.

FIGS. 2A to 2D show selected microscopic images of neural progenitor cells grown on exemplary peptide-modified polymer surfaces.

FIGS. 3A to 3D show microscopic images of neural progenitor stem cells on the peptide-modified polymer surfaces.

FIG. 4A shows a microscopic image of growth undifferentiated neural progenitor stem cells on laminin.

FIG. 4B shows a microscopic image of growth differentiation of neural progenitor stem cells on laminin. FIG. 5 show day 6 immuno-staining of differentiated neural progenitor cells on dEMA surfaces modified with selected collagen-peptide sequences and a laminin control.

FIG. 6 shows images of cell viability of primary hepatocytes from donors 817 and HC5-1 cultured on Collagen I (with and without serum) and hydrolyzed dEMA (no peptide attached) control surfaces evaluated by Live/Dead staining.

FIG. 7 shows images of cell viability of primary hepatocytes from donors 817 and HC5-1 cultured serum-free on dEMA surfaces conjugated with collagen peptides 10 and 11 evaluated by Live/Dead staining.

FIG. 8 shows day seven images of cell viability of primary hepatocytes from donors 817 and HC5-1 cultured serum-free on dEMA surfaces conjugated with collagen peptides 7, 8, and 9 evaluated by Live/Dead staining.

FIG. 9 shows day seven images of cell viability of primary hepatocytes from donors 817 and HC5-1 cultured serum-free on dEMA surfaces conjugated with collagen peptides 1, 2 and 3 evaluated by Live/Dead staining.

FIG. 10 shows 24 hr cell number data of primary hepatocytes from donor 817 in serum-free media cultured on dEMA surfaces conjugated with collagen peptides.

FIG. 11 shows data for seven day cell number of primary hepatocytes from donor 817 in serum-free media cultured on dEMA surfaces conjugated with collagen peptides.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

### DEFINITIONS

Peptide sequences are referred to herein by their one letter amino acid codes and by their three letter amino acid codes.

“Peptide” or like terms refer to an amino acid sequence that can be chemically synthesized or can be obtained recombinantly, or other than isolated as entire proteins from animal sources. The disclosed peptides are not whole proteins. Peptides can include amino acid sequences that are fragments of proteins. For example peptides can include sequences known as cell adhesion sequences such as RGD. Peptides can be of any suitable length, such as between three and thirty amino acids in length. Peptides can be, for example, acetylated (e.g., Ac-LysGlyGly) or amidated (e.g.SerLysSer-NH₂) to protect them from break down by, for example, exopeptidases. These modifications are contemplated when a sequence is mentioned.

“dEMA,” “derivatized EMA,” “derivatized ethylene-maleic anhydride copolymer,” or like terms refer to an EMA polymer which has been pre-blocked with at least one of various exemplary agents, such as ethanol amine or methoxyethyl amine, see commonly owned and assigned U.S. Pat. No. 7,781,203.

“RGD” refers to arginylglycylaspartic acid, a tripeptide composed of L-arginine, glycine, and L-aspartic acid, and to the tripeptide within larger entities such as polypeptides.

“Include,” “includes,” or like terms means encompassing but not limited to, that is, inclusive and not exclusive.

“About” modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example: through typical measuring and handling procedures used for making compounds, compositions, composites, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. The appended claims include equivalents of these “about” quantities.

“Consisting essentially of” in embodiments refers, for example, to a sol-gel polymer composition, to a method of making or using the hybrid sol-gel polymer composition, or formulation, and articles, devices, or any apparatus of the disclosure, and can include the components or steps listed in the claim, plus other components or steps that do not materially affect the basic and novel properties of the compositions, articles, apparatus, or methods of making and use of the disclosure, such as particular reactants, particular additives or ingredients, a particular agents, a particular surface modifier or condition, or like structure, material, or process variable selected. Items that can materially affect the basic properties of the components or steps of the disclosure or that can impart undesirable characteristics to the present disclosure include, for example, cell culture media which cannot provide exemplary growth and differentiation of selected cells or their progenitors.

The indefinite article “a” or “an” and its corresponding definite article “the” as used herein means at least one, or one or more, unless specified otherwise.

Abbreviations, which are well known to one of ordinary skill in the art, can be used (e.g., “h” or “hr” for hour or hours, “g” or “gm” for gram(s), “mL” for milliliters, and “rt” for room temperature, “nm” for nanometers, and like abbreviations).

Other abbreviation, such as the alphabet of single letter or three letter representations for an amino acid or combinations thereof for a peptide sequence are readily apparent to one of ordinary skill in the art and can be found, for example, in Lehninger, Principles of Biochemistry, 5^{th} Ed.,© 2009.

Specific and preferred values disclosed for components, ingredients, additives, and like aspects, and ranges thereof, are for illustration only; they do not exclude other defined values or other values within defined ranges. The compositions, apparatus, and methods of the disclosure can include any value or any combination of the values, specific values, more specific values, and preferred values described herein.

Brain tissue regeneration was not believed to be possible two decades ago (P. Rakic, “Limits of neurogenesis in primates,” Science, (1985), Vol. 227, No. 4690, 1054-1056). Recent work has confirmed that neurogenesis does occur in the adult human brain (P. S. Eriksson, et al., “Neurogenesis in the adult human hippocampus,” Nat. Med., (1998), Vol. 11, No. 13, 13-17; D. Kornack, et al., “Continuation of neurogenesis in the hippocampus of the adult macaque monkey,” Proc. Natl. Acad. Sci. U.S.A, (1999), Vol. 96, No. 10, 5768-5773; N. Sanai, et al., “Unique astrocyte ribbon in adult human brain contains neural stem cells but lacks chain migration,” Nature, (2004), Vol. 427, No. 6976, 740-744). Researchers use neurons and glia generated from the ectodermal germ layer of human embryonic stem cell (hESC) lines like H9, H7, H1, BG01, BG02, BG03, HES-1, HES-2, and HES-3, or from neural stem cells (NSC) or neural progenitor cells (NPC's) isolated from mouse or rat brain tissue, to understand the neural growth, differentiation, and for transplantation into the animal models, and to study neurodegenerative diseases (L. M. Hoffman, et al., “Characterization and culture of human embryonic stem cells,” Nat. Biotechnol., (2005), Vol. 23, No. 6, 699-708).

Maintenance and differentiation of stem cells into a particular subtype of neurons is a challenge and researchers have used several strategies in an attempt to unravel the mystery. Growth-factors-controlled-conditioned-media-supplementation and gene transfection are commonly used strategies to grow and differentiate neural stem cells (M. A. Caldwell, et al., “Growth factors regulate the survival and fate of cells derived from human neurospheres,” Nat. Biotechnol., (2001), Vol. 19, No. 5, 475-479; J. H. Kim, et al., “Dopamine neurons derived from embryonic stem cells function in an animal model of Parkinson's disease,” Nature, (2002), Vol. 418, No. 6893, 50-56; I. Liste, et al., “Bc1-XL modulates the differentiation of immortalized human neural stem cells,” Cell Death Differ., (2007), Vol. 14, No. 11, 1880-1892; Y. Hirabayashi, et al., “The Wnt/beta-catenin pathway directs neuronal differentiation of cortical neural precursor cells,” Development, (2004), Vol. 131, No. 12, 2791-2801; K. C. Sonntag, et al., “Enhanced yield of neuroepithelial precursors and midbrain-like dopaminergic neurons from human embryonic stem cells using the bone morphogenic protein antagonist noggin,” Stem Cells, (2007), Vol. 25, No. 2, 411-418).

Most of the neural stem cell culture has been accomplished on laminin coated surfaces (R. Donato, et al., “Differential development of neuronal physiological responsiveness in two human neural stem cell lines,” BMC Neurosci., (2007), Vol. 8, pg. 36; J. Gong, et al., “Effects of extracellular matrix and neighboring cells on induction of human embryonic stem cells into retinal or retinal pigment epithelial progenitors,” Exp. Eye Res., (2008), Vol. 86, No. 6, 957-965). In some instances poly L-lysine has also been used (I. Liste, et al., ibid.), or mixtures of polyornithine/laminin or poly D-lysine/laminin (K. C. Sonntag, et al., “Enhanced yield of neuroepithelial precursors and midbrain-like dopaminergic neurons from human embryonic stem cells using the bone morphogenic protein antagonist noggin,” Stem Cells, (2007), Vol. 25, No. 2, 411-418; Y. Yan, et al., “Directed differentiation of dopaminergic neuronal subtypes from human embryonic stem cells,” Stem Cells, (2005), Vol. 23, No. 6, 781-790; A. J. Joannides, et al., “A scalable and defined system for generating neural stem cells from human embryonic stem cells,” Stem Cells, (2007), Vol. 25, No. 3, 731-737). Mouse ESC's grow better on UltraWeb® with larger colonies and differentiate with more neuritis than without a 3D surface (Joannides, et al., ibid.). Matrigel™ has also been used in some studies to grow NSC's (J. S. Draper, et al., “Surface antigens of human embryonic stem cells: changes upon differentiation in culture,” Journal Anat., (2002), Vol. 200(Pt 3), 249-258; A. J. LaGier, et al., “Inhibition of human corneal epithelial production of fibrotic mediator TGF-beta2 by basement membrane-like extracellular matrix,” Invest. Ophthalmol. Vis. Sci., (2007), Vol. 48, No. 3, 1061-1071). A polymer scaffold poly(lactic-co-glycolic acid)/poly(L-lactic acid) has also been used to grow mouse ESC's (S. Levenberg, et al., “Differentiation of human embryonic stem cells on three-dimensional polymer scaffolds,” Proc. Natl. Acad. Sci. U.S.A, (2003), Vol. 100, No. 22, 12741-12746).

Freshly prepared laminin coated surface has been established as a standard for culture and differentiation of neural progenitor stem cells (K. C. Sonntag, et al., “Enhanced yield of neuroepithelial precursors and midbrain-like dopaminergic neurons from human embryonic stem cells using the bone morphogenic protein antagonist noggin”, Stem Cells, (2007), Vol. 25, No. 2, 411-418). Laminin is a natural extracellular matrix (ECM) protein which contains the RGD adhesion sequences that bind specifically to the cell-surface integrin receptors. Therefore, laminin facilitates the specific adhesion of anchorage dependent cells to the surfaces. Unfortunately, laminin is animal derived (exact composition is variable and is unknown) and has disadvantages such as lot-to-lot variability, high processing cost, and the presence of xeno-pathogens. Due to its limited stability, laminin must also be freshly coated prior to neural progenitor cell culture. Our work also indicated that laminin coated plates stored for two months or more do not support the attachment, growth, and differentiation of neural progenitor cells. This is also true for commercially available pre-coated laminin culture vessels.

Polystyrene and glass coated with synthetic extracellular matrix (ECM) proteins containing a repetitive RGD sequences have been used for neuronal PC12 cell culture in serum-free conditions (H. Kurihara, et al., “Cell adhesion ability of artificial extracellular matrix proteins containing a long repetitive Arg-Gly-Asp sequence,” J. Bioscience and Bioengineering, (2005), Vol. 100, 82-87). The same has been demonstrated with synthetic RGD peptides on dextran and N-(2-hydroxypropyl)methacrylamide for neural implant applications (S. P. Massia, et al., “In vitro assessment of bioactive coatings for neural implant applications,” J. Biomedical materials research, Part A, (2004), Vol. 68, 177-186; G. W. Plant, et al., “Hydrogels containing peptide or aminosugar sequences implanted into the rat brain: influence on cellular migration and axonal growth,” Experimental Neurology, (1997), Vol. 143, 287-299). Other workers have disclosed the use of surfaces containing RGD peptide sequences derived from various sources to culture neuronal cells for biosensor (European Patent 1180162 B1 and U.S. Pat. No. 7,266,457 B1), tissue regeneration (U.S. Pat. Pub. 2006/0194721), and other culture applications (U.S. Pat. Pub. 2009/0162437 and PCT Pub. No. WO2007030469 A2).

Furthermore, the in vitro assessment of human drug safety remains a major challenge in drug discovery. Specifically, in vitro models that enable the early stage study of absorption, metabolism, distribution, excretion, and toxicity (ADME-TOX) of newly developed drugs can lead to decreased late stage failure rate and safe early-stage pharmacological profiling (see Prentis, R. A., et al., Br. J. Clin. Pharm., 1988, 25, 387; Lasser, K. E., et al., J. Am. Med. Assoc., 287, 2215). Traditionally, pre-clinical drug safety evaluation takes place in lab animals, and later in humans during clinical trials after millions have been spent for FDA approval. More recent in vitro models rely on monolayer culture of fresh or cryopreserved human hepatocytes (see Soars, M. G., et al., Chemico-Biological Interactions, 2008, 168, 2; Li A. P., Chemico-Biological Interactions, 2007, 168, 16; Lee, M. Y., Curr. Opin. Biotecnol, 2006, 17, 619).

Collagen with Matrigel™ overlay (media additive) is an industry standard platform for the culture of primary hepatocytes for extended periods of time (see Moghe, P. V., et al., Biomat, 1996, 17, 737).

A synthetic sandwich derived from a GRGDS-modified polyethylene terephthalate (PET) membrane (top support) and a galactosylated PET film has been used to achieve a more 3D hepatocyte monolayer culture (see Du Yanan, et al., “Synthetic sandwich culture of 3D hepatocyte monolayer”, Biomaterials, (2007), Vol 29(3), 290-301). Other synthetic surfaces with RGD peptides coated or immobilized on polystyrene dishes (see Ijima, H., Biochemical Engineering Journal, (2008), 40(2), 387-391; De Bartolo, L., et al., Biomaterials (2005), 26(21), 4432-4441; Hansen, L. K., et al., Molecular Biology of the Cell (1994), 5(9), 967-75; Rubin, K., et al., Cell, (1981), 24(2), 463-70; Mooney, D. J., et al., Materials Research Society Symposium Proceedings (1992), 252 (Tissue-Inducing Biomaterials), 199-204), acrylic acid-grafted polyethersulfone membranes (Koh, W. G., et al., Analytical Chemistry (2003), 75(21), 5783-5789), sugars (Park, K. H., Biotechnology Letters (2002), 24(17), 1401-1406; WO2007136354 A1 Bioactive Surface for Hepatocyte-Based Applications), thermal responsive surfaces (Park, K. H., et al., Bioscience, Biotechnology, and Biochemistry (2002), 66(7), 1473-1478), self assembling peptides (Navarro-Alvarez, N., et al., Cell transplantation (2006); WO2009140573 Collagen Peptide Conjugates and Uses Therefore), micro-patterned materials (WO 00/56375 Mineralization and Cellular Patterning on Biomaterial Surfaces; WO 98/51785 Co-cultivation of Cells in a Micropatterned Configuration), synthetic microfibers (US2004/0126402 Engineered scaffolds for promoting growth of cells) and others (WO 2004/041061 Substrates, devices and methods for cellular assays) have been used for hepatocyte culture.

Many available surfaces for cell culture provide non-specific attachment of cells for cell growth. While useful, such surfaces do not allow for biospecific cell adhesion and thus do not readily allow for tailoring of characteristics of the cultured cells. For example, due to non-specific interactions it can be difficult to maintain cells, such as stem cells, in a particular state of differentiation or to direct cells to differentiate in a particular manner.

Some currently available surfaces provide for bio-specific adhesion, but employ animal derived coatings such as collagen, laminin or gelatin and other animal derived components. Such animal derived coatings can have inherent disadvantages described above.

In embodiments, the disclosure provides synthetic peptides of the formula:

(XₐX_{b})PQVTRGDVFTMP(X_{c}X_{d}),

where Xₐ and X_{d} are primary amine containing moieties or like functional moieties for conjugation of the peptide to a target and conjugation to the microcarrier surface, respectively, and X_{b} and X_{c} are optional hydrophilic linker moieties. Xₐ or X_{b} can be, for example, a lysine or like molecules which can have at least one or multiple primary amine groups. X_{b} or X_{c} can be, for example, of an polypeptide such as (Gly)ₙ where n is from 1 to 10, or a poly(ethylene glycol) which provides a linker or spacer structure and function and does not have cell binding function. In embodiments, at least one of XₐX_{b} or X_{c}X_{d} can be present in the synthetic peptide. In embodiments, XₐX_{b} and X_{c}X_{d} can be present in the synthetic peptide. In embodiments, the AAⱼ peptide-modification source can be, for example, of the formula:

(XₐX_{b})PQVTRGDVFTMP

where Xₐ and X_{d}, and X_{b} and X_{c} are as defined above.

In embodiments, the AAⱼ peptide-modification source can be, for example, of the formula:

PQVTRGDVFTMP(X_{c}X_{d}),

where Xₐ and X_{d}, and X_{b} and X, are as defined above.

In embodiments, the disclosure provides cell culture surfaces which do not include animal-derived ingredients or additives and which provide cell culture conditions amenable for cell culture of anchorage dependent cells, including the culture of difficult-to-culture cells such as stem cells. A surface that supports the specific attachment, retention, and long term cell viability and function is particularly useful for such models. Having a defined composition to culture cells such as embryonic stem cells, neural stem cells, primary hepatocytes or other cells while maintaining pluripotency, undifferentiation or specific functions for extended periods of time (<10 passages or several days depending on the cell type) would be advantageous. There is value in culturing cells in media where the exact composition is known (chemically defined/serum-free) as internal studies of gene expression have shown that serum free cultures (having chemically defined media) show enhanced gene expression of hepatic specific function for multiple surfaces. In the case of hepatocytes, internal studies of gene expression have shown that serum free cultures (with chemically defined media) show enhanced gene expression of hepatic specific function for multiple surfaces. For stem cells that will be used for therapeutic purposes, extra-cellular matrix proteins derived from animals can introduce infective agents such as viruses or prions. These infective agents can be taken up by cells in culture and, upon the transplantation of these cells into a patient, can be taken up into the patient. Therefore, the addition of these factors in or on cell culture surfaces can introduce new disease even as they address an existing condition. In addition, these animal-derived additives or cell culture surface coatings can lead to significant manufacturing expense and lot-to-lot variability which are undesirable. Cell culture surfaces which are free of animal-derived ingredients or additives and which provide cell culture conditions amenable for cell culture, including the culture of difficult-to-culture cells such as embryonic stem cells would be particularly useful. Furthermore, having a synthetic surface coating that supports adhesion of cells and is compatible with a biosensor such as the dEMA surface for Epic® biochemical assay) can offer the potential for drug assays or ADME-TOX studies. Specifically, if one were interested in testing or studying drug hepatocyte toxicity by Epic® cell-based assay, a confluent collagen coating would be too thick and would limit the assay detection as the cell would be outside the detection limit of about 150 nm. Furthermore, a collagen coating that is too thin would be discontinuous and would likely not exhibit good cell adhesion.

In embodiments, the present disclosure provides synthetic peptide surfaces for culturing cells. In embodiments, the surfaces can be configured to support proliferation and maintenance of, for example, undifferentiated neural stem cells. In embodiments, the surfaces can be configured to maintain, for example, human primary hepatocytes.

In embodiments, this disclosure provides a maleic anhydride copolymer surface, for example, dEMA having RGD peptide sequences derived from collagen and laminin for neural progenitor stem cells or primary hepatocyte cell culture. An RGD peptide sequence, that is arginylglycylaspartic acid, is a tripeptide composed of L-arginine, glycine, and L-aspartic acid. The sequence is a common element in cellular recognition. Unexpectedly, not all RGD containing peptides that were prepared and tested supported neural progenitor or primary hepatocyte cell culture.

In embodiments, the synthetic peptide modified surfaces are formed, for example, by i.) treating such as coating a base substrate with a tie layer; ii.) coating the maleic anhydride polymer (e.g., dEMA) onto the tie layer modified base substrate; and iii.) conjugating the synthetic cell-adhesive peptide to the maleic anhydride polymer coated substrate by, for example, amide bond formation.

In embodiments, the disclosure provides one or more advantages over prior articles and systems for culturing cells. For example, synthetic peptide modified surfaces described herein have been shown to support cell adhesion without an animal derived biocoating limit the risk of pathogen contamination. This is especially relevant when cells are dedicated to cell therapies. The methods provide for the preparation of surfaces having a wide range of adhesive properties based on the peptide origin (e.g., collagen, laminin, fibronectin, entactin). Furthermore, synthetic peptide modified surfaces for cell culture can alternatively be used for biosensor surfaces, such as Corning, Inc., Epic® instrument, for cell culture and differentiation studies, or ADME/TOX screening of drugs. These and other advantages will be understood from the following detailed descriptions when read in conjunction with the accompanying drawings.

### 1. Synthetic Peptide Modified Surfaces

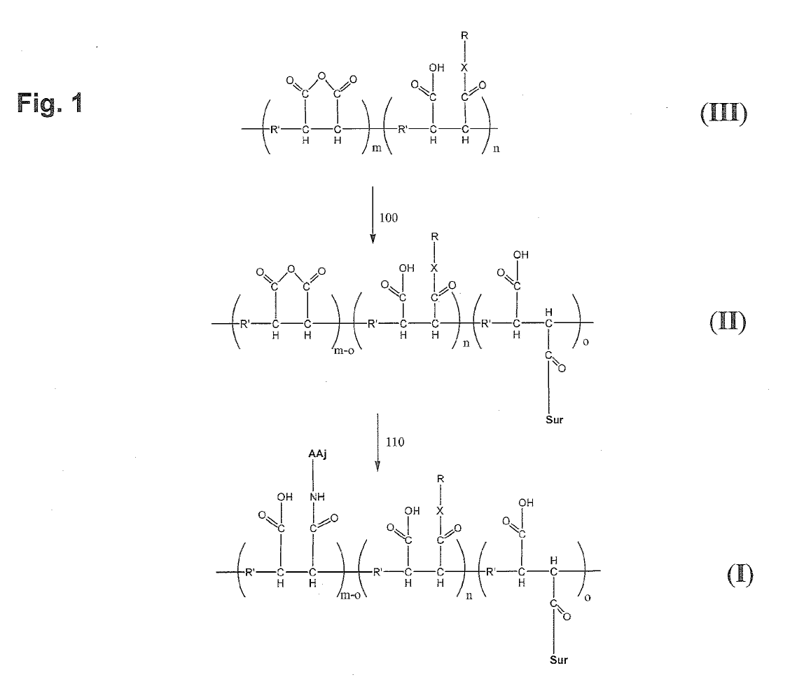

Referring to FIG. 1, the synthetic peptide modified surface includes an anhydride modified polymer coating of formula (I) having a covalent surface group (Sur) and a peptide group (AAj). The derivatized anhydride surface coating alone or in combination with the attached peptide can provide a surface to which cells can attach and can culture. In embodiments, the dEMA coating layer is deposited on or formed on a surface of an intermediate layer that is associated with the base material (Sur) via covalent or noncovalent interactions, either directly or via one or more additional intermediate layers or “tie layer(s)” (not shown). In such instances, the intermediate layer(s) can be considered as part of the synthetic peptide modified base surface.

In embodiments, the disclosure provides a maleic anhydride copolymer surface including, for example, i) dEMA having RGD peptide or like sequences derived from vitronectin, fibronectin, and bone sialoprotein, or collagen for neural progenitor cell culture; and ii) dEMA having RGD peptide or like sequences derived from collagen for primary human hepatocyte cell culture. An RGD peptide sequence, that is arginylglycylaspartic acid, is a tripeptide composed of L-arginine, glycine, and L-aspartic acid. The sequence is a common element in cellular recognition. Unexpectedly, not all RGD containing peptides that were prepared and tested supported neural progenitor cell culture, for example, Ac-KGGPQVTRGDVTMP-NH₂, GRGDSPK-NH₂, Ac-KGGAVTGRGDSPASS-NH₂, and Ac-KGGNGEPRGDTYRAY-NH₂ supported neural progenitor cell culture while, Ac-KGGGFRGDQ-NH₂, and Ac-KGGCKRARGDDMDDYC-NH₂ did not. Also, not all peptides that supported primary human hepatocytes contained RGD, for example, Ac-KGGCGGFHRRIKA-NH₂, and Ac-KGGGWKTSRTSHTC-NH₂ supported primary hepatocyte culture, while 7 other peptide sequences (sample ID #1-6 and 12) without RGD did not. In embodiments, the disclosure provides a stable synthetic surface having a well defined composition and structure that can support serum-free adhesion, long term proliferation, and differentiation of neural progenitor cells. In embodiments, the disclosure provides a stable synthetic surface having a well defined composition and structure that can support serum-free adhesion, long term culture of primary human hepatocytes.

### 2. Synthetic Peptide Modified Surfaces for Neural Stem Cells

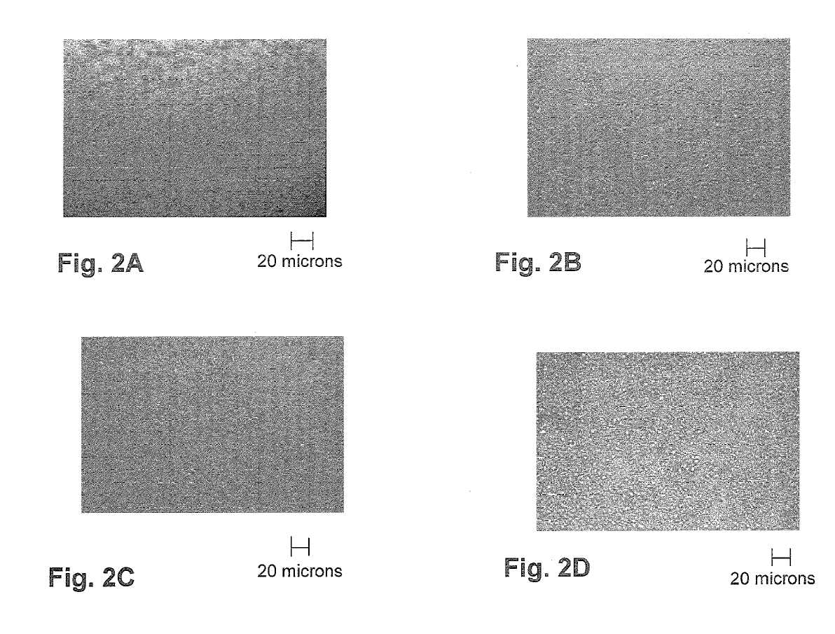

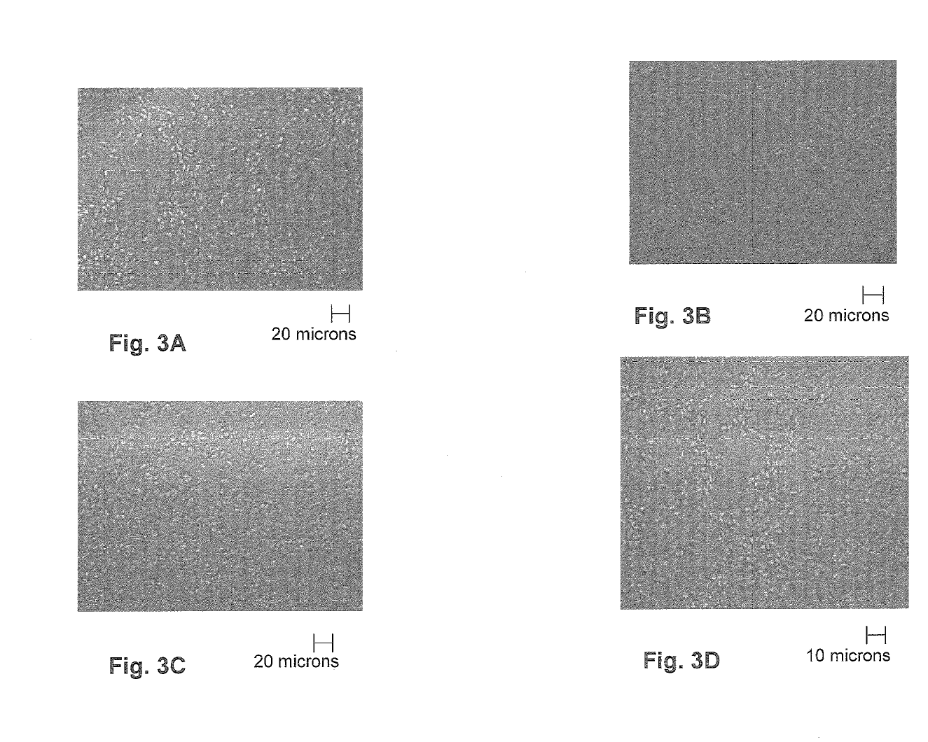

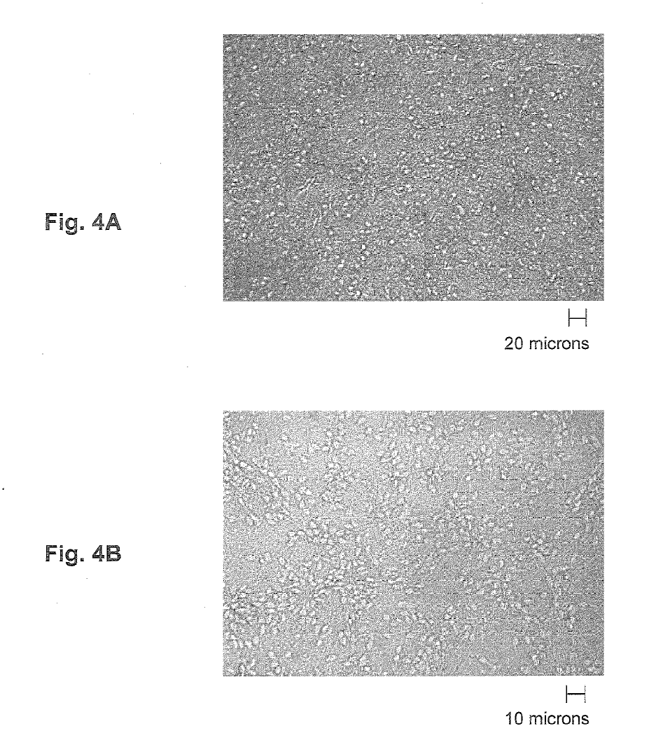

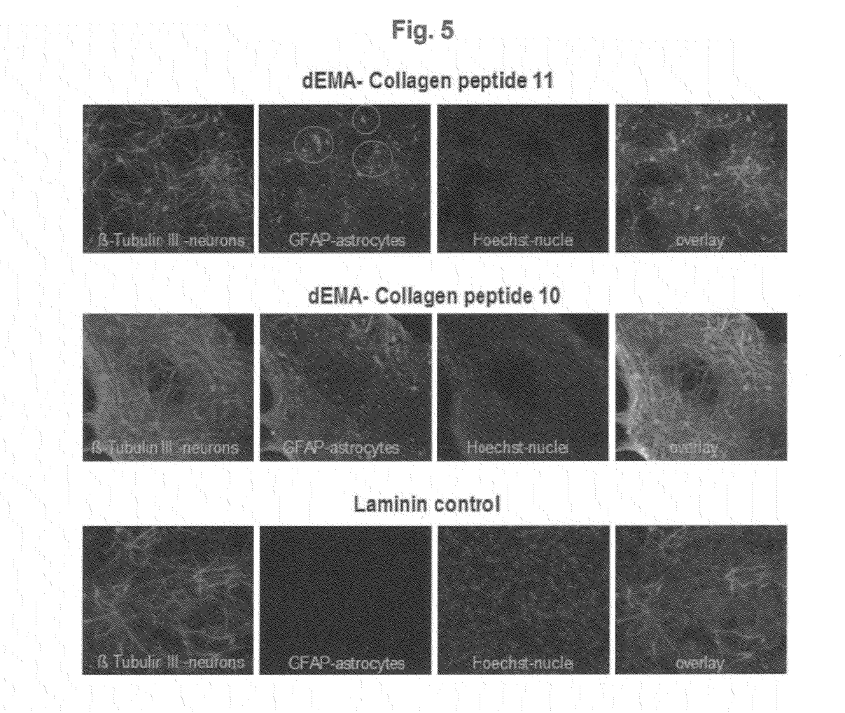

In embodiments, the disclosure provides a method for the preparation and use of synthetic laminin peptide-derived surfaces that can support serum-free culture of neural progenitor stem cells. The surfaces were prepared by direct conjugation of peptide sequences to a derivatized EMA surfaces. The peptides selected to mimic sequences of, for example, laminin derived from vitronectin Ac-KGGPQVTRGDVTMP-NH₂ (VN), fibronectin GRGDSPK (short FN), Ac-KGGAVTGRGDSPASS-NH₂ (long FN), and bone sialoprotein Ac-KGGNGEPRGDTYRAY-NH₂ (BSP). Alternatively, the laminin peptide-derived surfaces surface can be prepared with other maleic anhydride polymers. Of the sixteen peptide sequences that were selected to mimic sequences of laminin and tested (from several origins and some containing RGD, Table 1), the sequences Ac-KGGNGEPRGDTYRAY-NH₂ (BSP) and Ac-KGGPQVTRGDVFTMP-NH₂ (VN) showed superior proliferation (FIG. 2) and differentiation (FIG. 3) of neural progenitor cells under serum-free conditions relative to the other peptide sequences and comparable to the freshly prepared laminin control (FIG. 4) and as confirmed by phase contrast microscopy.

In embodiments, this disclosure provides a method for the preparation and use of synthetic collagen peptide-derived surfaces that can support serum-free culture of neural progenitor cells. The surface was prepared by direct conjugation of the collagen peptide sequence, Ac-KGGGRGDTP-NH₂, to derivatized EMA. Alternatively, the surface can be prepared with other maleic anhydride polymers. The twelve (12) collagen peptide sequences (from several sources and some containing RGD) listed in Table 2 were prepared and tested as a cell culture support. Only the Ac-KGGGRGDTP-NH₂ sequence supported the specific attachment, proliferation, differentiation, and further growth of neural cells under serum-free conditions similar to the freshly coated laminin control as confirmed by immuno-staining, FIG. 5. Furthermore, neural progenitor cells differentiated on Ac-KGGGRGDTP-NH₂ conjugated to dEMA appeared to yield more astrocytes than freshly coated laminin (FIG. 5), indicating an increased efficiency of astrocyte differentiation.

The synthetic laminin peptide sequences Ac-KGGNGEPRGDTRAY-NH₂ (BSP), GRGDSPK (short FN), Ac-KGGAVTGRGDSPASS-NH₂ (long FN), and Ac-KGGPQVTRGDVTMP-NH₂ (VN), have been identified as excellent surface modifiers for the culture of neural progenitor cells. When each of these peptide sequences was conjugated to a surface associated polymer, such as dEMA, they supported serum-free specific attachment, expansion, and differentiation of neural progenitor cells. The results for each conjugated peptide sequence were comparable to a freshly coated laminin surface. These surface can be a synthetic replacement for laminin.

The synthetic collagen peptide sequence Ac-KGGGRGDTP-NH₂ was identified as an excellent surface modifier for cell culture of neural progenitor cells. When this peptide sequence was conjugated to a surface associated polymer, such as dEMA, the resulting peptide modified surface supported serum-free specific attachment, expansion, and differentiation of neural progenitor cells. The results were comparable to a freshly coated laminin surface. This surface can also be a synthetic replacement for laminin. The maleic anhydride-laminin and collagen peptide-modified surfaces developed for neural progenitor cell culture can also be used as biosensor surfaces, such as Epic®, for use in culture, differentiation, and neurotoxicity studies, or to probe and analyze the molecular mechanisms and signaling pathways involved in neural progenitor cell growth and differentiation.

TABLE 1
Sequence, origin, and receptor identification of the laminin peptide
sequences used.

| Sample | | | |
|---|---|---|---|
| ID | Sequence | Source | Receptor(s) |
| 1 | KGGGQKCIVQTTSWSQCSKS-NH₂ | Cyr61 res 224-240 | α6β1 Integrin |
| 2 | KYGLALERKDHSG-NH₂ | TSP1 res 87-96 | α6β1 Integrin |
| 3 | KGGSINNNRWHSIYITRFGNMGS- | mLMα1 res 2179-2198 | |
| | NH₂ | | |
| 4 | KGGTWYKIAFQRNRK-NH₂ | mLMα1 res 2370-2381 | α6, α3, β1 |
| 5 | KGGTSIKIRGTYSER-NH₂ | mLMγ1 res 650-261 | α2 and α6, not |
| | | | β1 |
| 6 | KYGTDIRVTLNRLNTF-NH₂ | mLMγ1 res 245-257 | |
| 7 | KYGSETTVKYIFRLHE-NH₂ | mLMγ1 res 615-627 | |
| 8 | KYGKAFDITYVRLKF-NH₂ | mLMγ1 res 139-150 | |
| 9 | KYGAASIKVAVSADR-NH₂ | mLMα1 res 2122-2132 | HSPGs |
| 10 | Ac-KGGNGEPRGDTYRAY-NH₂ | Bonesialoprotein | |
| | | (BSP) | |
| 11 | Ac- KGGNGEPRGDTRAY-NH₂ | Bonesialoprotein | |
| | | (BSP-Y) | |
| 12 | KYGRKRLQVQLSIRT-NH₂ | mLMα1 res 2719-2730 | HSPGs |
| 13 | KGGRNIAEIIKDI-NH₂ | LMβ1 | |
| 14 | Ac-KGGPQVTRGDVFTMP-NH₂ (VN) | Vitronectin | |
| 15 | GRGDSPK-NH₂ (short FN) | Fibronectin | |
| 16 | Ac-KGGAVTGRGDSPASS-NH₂ | Fibronectin | |
| | (long FN) | | |

### 3. Synthetic Peptide Modified Surfaces for Human Primary Hepatocytes

In embodiment, this disclosure provides methods of making and using several synthetic collagen peptide-derived surfaces that support serum-free culture of primary human hepatocytes. The surfaces were prepared by direct conjugation of collagen peptide sequences of various origins to maleic anhydride bearing surfaces, such as derivatized EMA (dEMA). Of the 12 collagen peptide sequences that were separately conjugated to a dEMA surface and tested (see Table 2), several did not support cell retention beyond 24 hr as cell loss with media change was observed over time. However, peptides 10 (Ac-KGGCKRARGDDMDDYC-NH₂), 11 (Ac-KGGGRGDTP-NH₂), 7 (Ac-KGGGFRGDGQ-NH₂), 8 (Ac-KGGCGGFHRRIKA-NH₂), and 9 (Ac-KGGGWKTSRTSHTC-NH₂) all supported specific attachment, retention, cell viability, and morphology over a seven day period as confirmed using an MTS assay and Live/Dead fluorescent staining In particular, collagen peptides 10 and 11, sequences Ac-KGGCKRARGDDMDDYC-NH₂ and Ac-KGGGRGDTP-NH₂, respectively, when conjugated to dEMA, supported the best cell attachment and cell retention. The primary hepatocytes were from at least two different human donors (817 and HC5-1) and formed a confluent monolayer similar to the commercial standard surface, Collagen I.

TABLE 2
Sequence, origin, and receptor identification of
collagen peptide sequence used.

| Sample | | | |
|---|---|---|---|
| ID | Sequence | Source | Receptor(s) |
| 1 | Ac-KGGCGGDGEAG-NH₂ | — | alpha2beta1(α2β1) |
| 2 | Ac-KGGCWKTSLTSHTC-NH₂ | obstutatin | Alpha1beta1(α1β1) + |
| | | | alpha1beta2(α1β2) |
| 3 | Ac-KGGGASGERGPO-NH₂ | bovine | α1β1 + α1β2 |
| 4 | Ac-KGGGLOGERGRO-NH₂ | bovine | α1β1 + α1β2 |
| 5 | Ac-KGGGFOGERGVQ-NH₂ | bovine | α1β1 + α1β2 |
| 6 | Ac-TAGSCLRKFSTMGGK-NH₂ | — | α1β1 + α1β2 |
| 7 | Ac-KGGGFRGDGQ-NH₂ | entactin | — |
| 8 | Ac-KGGCGGFHRRIKA-NH₂ | — | — |
| 9 | Ac-KGGGWKTSRTSHTC-NH₂ | viperistatin | α1β1 + α1β2 |
| 10 | Ac-KGGCKRARGDDMDDYC- | echistantin | α1β1 + α1β2 |
| | NH₂ | | |
| 11 | Ac-KGGGRGDTP-NH₂ | — | — |
| 12 | Ac-KGGGPOGFOGERGPO-NH₂ | — | α1β1 + α1β2 + α1β |

O = hydroxyproline; Ac = acetyl

The maleic anhydride-collagen peptide-modified surfaces developed for primary hepatocyte cell culture can also be used as biosensor surfaces, such as Epic®, for use in culture, ADME-TOX assay detection, or to probe and analyze the molecular mechanisms and signaling pathways involved in hepatocyte culture and function. Specifically, if one were interested in testing or studying drug hepatocyte toxicity by Epic® cell-based assay, a confluent collagen coating would be too thick and would limit the assay detection as the cell would be outside the detection limit of about 150 nm. Furthermore, a collagen coating that is too thin would be discontinuous and would likely not exhibit good cell adhesion. Additionally or alternatively, the maleic anhydride-collagen peptide modified surfaces can be coated onto low density glass beads and used for three dimensional (3D) suspension culture of primary hepatocytes for other functional studies. In embodiment, the disclosure provides synthetic collagen peptide-modified surfaces having defined and known composition, that support serum-free attachment, long term retention, cell viability, and morphology of hepatocytes comparable to Collagen I. This surface can be a synthetic replacement for Collagen I surface and Collagen-Matrigel™ sandwich currently used for primary hepatocyte culture.

In embodiments of the present invention, maleic anhydride peptide-modified surfaces that impart specific physical and chemical attributes to the surface, and methods of making these surfaces are provided. These specific physical and chemical attributes can facilitate the proliferation difficult-to-culture cells such as stem cells in embodiments of the present invention. These maleic anhydride peptide-modified surfaces can be prepared with different properties. The maleic anhydride polymer, peptide conjugate and blocking agents have particular characteristics which, when combined and coated provide maleic anhydride peptide-modified surfaces that are amenable for cell culture. These characteristics can include hydrophilicity or hydrophobicity, positive charge, negative charge or no charge, and compliant or rigid surfaces. For example, blocking agents or combinations of peptides which are hydrophilic can provide cell culture surfaces that are preferable in embodiments of the present invention. Alternatively, blocking agents or combinations of peptides which carry a charge can be preferable in embodiments of the present invention. Alternatively, blocking agents or combinations of peptides which influence swelling of the maleic anhydride polymer can influence certain range of modulus or hardness can be useful in embodiments of the disclosure. Alternatively, monomers or blocking agents or combinations of peptides which exhibit a combination of these attributes can be preferable in embodiments of the disclosure.

Surfaces for cell culture can be described according to their characteristics such as hydrophobicity, hydrophilicity, surface charge or surface energy, wettability or contact angle, topography, modulus which describes the surface's stiffness versus compliance, as well as chemical characteristics such as the surface expression of active chemical moieties such as oxygen or nitrogen.

### 4. Base Substrate

Any suitable base substrate can be used. “Base substrate” includes substrate, base, and like terms. The substrates that can be used include, for example, a microplate, a slide, or any other material that is capable of attaching to the binding polymer. In embodiments, when the substrate is a microplate, the number of wells and well volume can vary depending upon the scale and scope of the analysis. Other examples of useful substrates include, for example, a cell culture surface such as a 384-well microplate, a 96-well microplate, 24-well dish, 8-well dish, 10 cm dish, or a T75 flask.

For optical or electrical detection applications, the substrate can be transparent, impermeable, or reflecting, and electrically conducting, semiconducting, or insulating. For biological applications, the substrate material can be either porous or nonporous and can be selected from either organic or inorganic materials.

In embodiments, the substrate can be a plastic, a polymeric or co-polymeric substance, a ceramic, a glass, a metal, a crystalline material, a noble or semi-noble metal, a metallic or non-metallic oxide, an inorganic oxide, an inorganic nitride, a transition metal, and like materials, or any combination thereof. Additionally, the substrate can be configured so that it can be placed in any detection device. In embodiments, sensors can be integrated into the bottom or underside of the substrate and used for subsequent detection. These sensors could include, for example, optical gratings, prisms, electrodes, and quartz crystal microbalances. Detection methods can include, for example, fluorescence, phosphorescence, chemiluminescence, refractive index, mass, electrochemical. In embodiments, the substrate can be a resonant waveguide grating sensor.

In embodiments, the substrate can be, for example, an inorganic material. Examples of inorganic substrate materials include, for example, metals, semiconductor materials, glass, and ceramic materials. Examples of metals that can be used as substrate materials include, for example, gold, platinum, nickel, palladium, aluminum, chromium, steel, and gallium arsenide. Semiconductor materials can be used for the substrate material include, for example, silicon and germanium. Glass and ceramic materials can be used for the substrate material and can include, for example, quartz, glass, porcelain, alkaline earth aluminoborosilicate glass and other mixed oxides. Further examples of inorganic substrate materials include graphite, zinc selenide, mica, silica, lithium niobate, and inorganic single crystal materials. In embodiments, the substrate can be made of gold such as, for example, a gold sensor chip.

For hydroxyl containing inorganic substrates, factors such as initial concentration of surface hydroxyls, type of surface hydroxyls, stability of the bond formed and dimensions or features of the substrate can influence the effectiveness of the tie layer or polymer coating. It can be desirable to have the maximum number of accessible reactive sites on the glass surface to maximize initiator coupling. Acid or base etching (e.g., 1M sodium hydroxide, ammonia, hydrochloric acid), UV-ozone, or plasma treatment can be included as a step to pretreat the glass surface to clean, expose, or both to the more reactive silanol groups which can interact with the silane-initiator. Other hydroxyl-containing substrates such as silica, quartz, aluminum, alumino-silicates, copper inorganic oxides, etc. can be used as an alternative to glass.

In embodiments, the substrate can be a porous, inorganic layer. Any of the porous substrates and methods of making such substrates disclosed in U.S. Pat. No. 6,750,023, can be used. In embodiments, the inorganic layer on the substrate can be a glass or metal oxide. In embodiments, the inorganic layer can be a silicate, an aluminosilicate, a boroaluminosilicate, a borosilicate glass, or a combination thereof. In embodiments, the inorganic layer comprises TiO₂, SiO₂, Al₂O₃, Cr₂O₃, CuO, ZnO, Ta₂O₅, Nb₂O₅, ZnO₂, or a combination thereof. In embodiments, the substrate can be SiO₂ with a layer comprising Ta₂O₅, Nb₂O₅, TiO₂, Al₂O₃, silicon nitride or a mixture thereof, wherein the layer can be adjacent to the surface of the SiO₂. The silicon nitride can be represented by the formula SiN_{X}, where the stoichiometry of silicon and nitrogen can vary.

In embodiments, the substrate can be an organic material. Useful organic materials can be made from polymeric materials due to their dimensional stability and resistance to solvents. Examples of organic substrate materials include, for example, polyesters, such as poly(ethylene terephthalate) and poly(butyleneterephthalate); poly(vinylchloride); poly(vinylidene fluoride); poly(tetrafluoroethylene); polycarbonate; polyamide; poly(meth)acrylate; polystyrene, polyethylene, ethylene/vinyl acetate copolymer, and like polymers, or mixtures thereof.

The base surface can be porous or non-porous. “Non-porous” means having no pores or pores of an average size smaller than a cell on which the surface it is cultured, e.g., less than about 0.5 to about 1 micrometers. Non-porous surfaces can be desired when the surface is not degradable, because cells that enter pores of macroporous base can be difficult to remove. However, if the base surfaces are degradable, e.g., if they include an enzymatically or otherwise degradable cross-linker, it can be desirable for the surfaces to be macroporous.

### 5. Binding Polymer

In embodiments, a binding polymer comprising one or more reactive groups that can bind a peptide to the substrate can be directly or indirectly attached to the substrate. The “reactive group” on the binding polymer permits the attachment of the binding polymer to the peptide. The reactive groups can also facilitate the attachment of the binding polymer to the substrate. In embodiments, the binding polymer can be either or both covalently, or electrostatically attached to the substrate. The binding polymer can have one or more different reactive groups. It is also contemplated that two or more different binding polymers can be attached to the substrate.

In embodiments, the reactive group is capable of forming a covalent bond with a nucleophile such as, for example, an amine or thiol. The amine or thiol can be derived from the biomolecule or a molecule that is attached to the surface of the substrate (i.e., a tie layer) and used to indirectly attach the binding polymer to the substrate. Examples of reactive groups include, for example, an anhydride group, an epoxy group, an aldehyde group, an activated ester (e.g., N-hydroxysuccinimide (NHS)), an isocyanate, an isothiocyanate, a sulfonyl chloride, a carbonate, an aryl or alkyl halide, an aziridine, or a maleimide. It is contemplated that two or more different reactive groups can be present on the binding polymer.

In embodiments, the binding polymer can be a synthetic coating free from animal-derived components, since animal derived components may contain viruses or other infectious agents or can provide a high level of batch-to-batch variability. In embodiments, the coating can be a maleic anhydride based coating, see for example, the above mentioned commonly owned and assigned U.S. Pat. No. 7,781,203.

Also present on the binding polymer can be a plurality of ionizable groups. Ionizable groups refer to groups that can be converted to a charged (i.e., ionic) group under particular reaction conditions. For example, a carboxylic acid (an ionizable group) can be converted to the corresponding carboxylate (negatively charged group) by treating the acid with a base. The charged groups can be either positive or negative. An example of a positively charged group is an ammonium group. Examples of negatively charged groups include carboxylate, sulfonate, and phosphonate groups. It is contemplated that two or more different ionizable groups can be present on the binding polymer.

The binding polymer can be water-soluble or water-insoluble depending upon the technique used to attach the binding polymer to the substrate. The binding polymer can be either linear or non-linear. For example, when the binding polymer is non-linear, the binding polymer can be branched, hyperbranched, crosslinked, or dendritic polymer. The binding polymer can be a homopolymer or a copolymer.

In embodiments, the binding polymer comprises a copolymer comprises of maleic anhydride monomers and a first monomer. The amount of maleic anhydride in the binding polymer can be, for example, from 5% to 50%, 5% to 45%, 5% to 40%, 5% to 35%, 5% to 30%, 5% to 25%, 10% to 50%, 15% to 50%, 20% to 50%, 25% to 50%, or 30% to 50 mol % or by stoichiometry (i.e., relative mole amount) of the first monomer, including intermediate values and ranges. In embodiments, the first monomer selected can improve the stability of the maleic anhydride group in the binding polymer. The first monomer can reduce nonspecific binding of the biomolecule to the substrate. The amount of maleic anhydride in the binding polymer can be, for example, about 50 mol % of the first monomer. The first monomer can be, for example, styrene, tetradecene, octadecene, methyl vinyl ether, triethylene glycol methyl vinyl ether, butylvinyl ether, divinylbenzene, ethylene, dimethylacrylamide, vinyl pyrrolidone, a polymerizable oligo(ethylene glycol) or oligo(ethylene oxide), propylene, isobutylene, vinyl acetate, methacrylate, acrylate, acrylamide, methacrylamide, or a combination thereof. The binding polymer can be, for example, poly(vinylacetate-co-maleic anhydride), poly(styrene-co-maleic anhydride), poly(isobutylene-alt-maleic anhydride), poly(maleic anhydride-alt-1-octadecene), poly(maleic anhydride-alt-1-tetradecene), poly(maleic anhydride-alt-methyl vinyl ether), poly(triethyleneglycol methlyvinylether-co-maleic anhydride), poly(ethylene-alt-maleic anhydride), or a combination thereof.

6. Coating of Base Substrate with Binding Polymer

Binding polymers are brought in contact with the functionalized base substrate. In embodiments, the base is referred to as the “substrate” on which the tie layer or binding polymer is deposited or formed. By way of example, the substrate can be suspended or dipped in the binding polymer solution and the substrate can be coated with the binding polymer through covalent reaction. As the binding polymer can be in form of a solid or viscous liquid, it can be desirable to dilute the binding polymer in a suitable solvent to reduce viscosity prior to suspending the binding polymer with the base substrate. Reducing viscosity can allow for thinner and more uniform layers of the coating material to be formed. The solvent is compatible with the base material and the binding polymer. It can be desirable to select a solvent that is nontoxic to the cells to be cultured and that does not interfere with the coating reaction. Alternatively, or additionally, selection of a solvent that can be substantially completely removed or removed to an extent that it is non-toxic or no longer interferes with coating can be desirable. In such circumstances, it can be desirable that the solvent be readily removable without harsh conditions, such as high vacuum or extreme heat. Volatile liquids are examples of such readily removable solvents.

Some solvents that can be suitable in various situations for coating the described articles include N-methyl-2-pyrrolidone (NMP), dimethylformamide (DMF), dimethylsulfoxide (DMSO), 2-propanol (IPA), methanol, ethanol, acetone, butanone, acetonitrile, 2-butanol, acetyl acetate, ethyl acetate, water or combinations thereof. In embodiments, it can desirable that the binding polymer is inert to the solvent and does not hydrolyze or react with the binding polymer.

The binding polymer can be diluted with solvent by any suitable amount to achieve the desired viscosity, binding polymer concentration, or colloidal suspension. For example, the binding polymer solution can contain between about 0.1% to about 99% binding polymer. For example, the binding polymer can be diluted with an ethanol or like solvents to provide a composition having between about 0.1% and about 50% monomer, or from about 0.1% to about 10% binding polymer by volume, or from about 0.1% to about 1% binding polymer by volume, and like concentrations including intermediate values and ranges. The polymer can be diluted with solvent so that the coating layer achieves a desired thickness.

The binding polymer can be coated as a colloidal solution. A colloidal solution can be created by first dissolving the binding polymer into highly compatible solvent and allowing it to fully dissolve, followed by dilution with a poor solvent that can partially precipitate the polymer from the solution.

The substrate bound binding polymer coating can be washed with solvent one or more times to remove impurities such as unbound polymer or low molecular weight polymer species. In various embodiments, the layer is washed with ethanol, greater than 90% ethanol, greater than 95% ethanol or greater than about 99% ethanol. Preferably, the wash solvent does not contain any water or nucleophilic species that can hydrolyze the unreacted reactive groups within the binding polymer. Hydrolysis can render the resulting surface unreactive towards a desired peptide. The size and shape of the base substrate can determine washing method. For example, a flat sheet can be washed by dipping in solvent or washed by squirt bottle, spraying, or any other washing methods. Any suitable filter apparatus can be incorporated to remove the washing solvent of microparticle substrates. Examples of filter systems are peptide synthesis vessels equipped with a vacuum filter or a Soxhlet apparatus for higher temperature washings.

Useful binding polymers are those that do not contain a photoreactive group. Photoreactive groups respond to specific applied external stimuli to undergo active species generation with resultant covalent bonding to an adjacent chemical structure, e.g., as provided by the same or a different molecule. Photoreactive groups are those groups of atoms in a molecule that retain their covalent bonds unchanged under conditions of storage; however, upon activation by an external energy source, form covalent bonds with other molecules. The photoreactive groups can generate active species such as free radicals and particularly nitrenes, carbenes, and excited states of ketones upon absorption of electromagnetic energy.

### 7. Ratio of Reactive Groups to Ionizable Groups

In embodiments, the ratio of reactive groups to ionizable (i.e., ionic) groups can be, for example, from 0.5 to 5.0. In embodiments, the lower endpoint of the ratio can be, for example, 0.5, 0.6, 0.7, 0.8, 0.9, 1.0, 1.5, 2.0, 2.5, 3.0, 3.5, 4.0, 4.5, or 5.0, and the upper endpoint is 2.0, 2.5, 3.0, 3.5, 4.0, 4.5, 5.0, 5.5, 6.0, 6.5, 7.0, 7.5, 8.0, 8.5, 9.0, 9.5, or 10.0, including intermediate values and ranges, where any lower and upper endpoint can form the ratio range. In embodiments, the ratio of reactive groups to ionizable groups can be, for example, from 0.5 to 9.0, 0.5 to 8.0, 0.5 to 7.0, 0.5 to 6.0, 0.5 to 5.0, 0.5 to 4.0, 0.5 to 3.0, 0.6 to 3.0, 0.65 to 3.0, or 0.67 to 3.0, including intermediate values and ranges.

The formation and number of reactive groups and ionizable groups present on the binding polymer can be controlled in a number of ways. In embodiments, the binding polymer can be synthesized from monomers possessing reactive groups and monomers with ionizable groups. In this aspect, the stoichiometry of the monomers selected can control the ratio of reactive groups and ionizable groups. In embodiments, a polymer possessing just reactive groups can be treated so that some of the reactive groups are converted to ionizable groups prior to attaching the binding polymer to the substrate. The starting polymer can be commercially available or synthesized using techniques known in the art. In embodiments, a polymer can be attached to the substrate, and the attached polymer can be treated with various reagents to add either reactive groups and ionizable groups or convert reactive groups to ionizable groups. In embodiments, the binding polymer that possesses reactive groups can be attached to the substrate, where the substrate reacts with the reactive groups and produces ionizable or ionic groups.

For example, referring to FIG. 1, when a polymer with a repeat unit of R′-maleic anhydride, where R′ can be a residue of an unsaturated monomer selected among monomers able to copolymerize with maleic anhydride such as, for example, ethylene, propylene, isobutylene, octadecene, tetradecene, vinyl acetate, styrene, vinyl ethers such as methyl vinyl ether, butyl vinyl ether, triethylene glycol vinyl ether, (meth)acrylates, (meth)acrylamide, vinyl pyrrolidinone, polymerizable oligo(ethylene glycol) or oligo(ethylene oxide) is reacted with X—R, where X is a nucleophilic group such as, for example, NH₂, OH, or SH and R can be hydrogen or a substituted or unsubstituted alkyl group (linear or branched) having from 1 to 6 carbon atoms, an oligo(ethylene oxide) or oligo(ethylene glycol), or a dialkyl amine such as dimethyl amino propyl or diethyl amino propyl, the anhydride ring-opens and produces the carboxylic acid (an ionizable group) to form the polymer of formula (III). This step is referred to as pre-blocking. The pre-blocked polymer can then be applied to the surface of the substrate. Referring to FIG. 1, if the substrate possesses nucleophilic surface or substrate groups (Sur), where Sur can be, for example, NH₂, OH, or SH, these groups can react with the maleic anhydride groups present on the pre-blocked polymer to form a covalent bond between the pre-blocked polymer and the substrate of the formula (II).

The ratio of reactive groups to ionizable groups can be controlled by using specific amounts of reagents. Other properties of the binding polymer (e.g., hydrophobicity) can be altered as needed by controlling the starting materials used to prepare the binding polymer (e.g., selection of hydrophobic monomers) or by appropriate choice of the derivatizing/blocking/pre-blocking reagent. In embodiments, the ratio of reactive groups to ionizable groups can be controlled by converting the one or more reactive groups to inactive groups. In a further aspect, from about 10% to about 50% of the reactive groups present on the binding polymer can be blocked or rendered inactive. “Blocked” refers to the conversion of a reactive group present on the binding polymer to an inactive group, where the inactive group does not form a covalent attachment with a biomolecule. In various aspects, the amount of reactive groups that are blocked can be, for example, about 10%, about 12%, about 14%, about 16%, about 18%, about 20%, about 22%, about 24%, about 26%, about 28%, about 30%, about 32%, about 34%, about 36%, about 38%, about 40%, about 42%, about 44%, about 46%, about 48%, or about 50% relative mole percent, including intermediate values and ranges, where any value can form a lower and upper endpoint of a range. In embodiments, from about 10% to about 45%, 10% to about 40%, 10% to about 35%, 15% to about 35%, 20% to about 35%, or about 25% to about 35%, relative mole percent, including intermediate values and ranges, of the reactive groups are blocked.

The blocking agent can react with the binding polymer prior to attaching the binding polymer to the substrate or, alternatively, the binding polymer can be attached to the substrate first followed by blocking with the blocking agent. In a further aspect, the blocking agent comprises at least one nucleophilic group, the binding polymer comprises at least one electrophilic group, and the blocking agent is attached to the binding polymer by a reaction between the electrophilic group and the nucleophilic group. In embodiments, the blocking agent can be covalently attached to the binding polymer. For example, when the blocking agent comprises an amine group, hydroxyl group, or thiol group, it can react with an electrophilic group present on the binding polymer (e.g., an epoxy, anhydride, activated ester group) to produce a covalent bond.

In embodiments, the blocking agent comprises an alkyl amine, an alkylhydroxy amine, or an alkoxyalkyl amine. “Alkyl” refers to a branched or unbranched saturated hydrocarbon group of 1 to 25 carbon atoms, such as methyl, ethyl, n-propyl, isopropyl, n-butyl, isobutyl, t-butyl, pentyl, hexyl, tetracosyl, and like groups. Examples of longer chain alkyl groups include an oleate group or a palmitate group. A “lower alkyl” group is an alkyl group containing from one to six carbon atoms. “Alkylhydroxy” refers to an alkyl group as defined above where at least one of the hydrogen atoms is substituted with a hydroxyl group. “Alkylalkoxy” refers to an alkyl group as defined above where at least one of the hydrogen atoms is substituted with an alkoxy group-OR, where R is an alkyl group as defined above.

In embodiments, the blocking agent can be, for example, ammonia, 2-(2-aminoethoxy)ethanol, N,N-dimethyl ethylenediamine, ethanolamine, ethylenediamine, hydroxylamine, methoxyethyl amine, ethyl amine, isopropyl amine, butyl amine, propyl amine, hexyl amine, 2-amino-2-methyl-1-propanol, 2-(2-aminoethyl amino) ethanol, 2-(2-aminoethoxy)ethanol, dimethylethanolamine, dibutyl ethanolamine, 1-amino-2-propanol, polyethylene glycol, polypropylene glycol, 4,7,10-trioxa-1,13-tridecanediamine, polyethylene glycol or an amine-terminated-polyethylene glycol, Trizma hydrochloride, or any combination thereof. In another aspect, the blocking agent comprises water, H₂S, an alcohol (ROH), or alkyl thiol (RSH), where R is an alkyl group as defined above.

The disclosed supports having the ratio of reactive groups to ionizable groups present on the binding polymer possess numerous advantages over prior art sensors. The ratio of reactive groups to ionizable groups permits increased loading or attachment (directly or indirectly with the use of a tie layer) of the binding polymer to the substrate. The attachment of the binding polymer to the substrate involves mild conditions and does not require preactivation with, for example, EDC/NHS. This saves time and costs with respect to manufacturing the supports. It is also possible to control the ratio of reactive groups/ionizable groups with other properties of the binding polymer such as hydrophobicity/hydrophilicity, which can increase the efficiency of the support.

Another feature of the disclosed supports is the higher binding capacity between the support and the biomolecule. It is believed that if more binding polymer can be loaded on the substrate then more biomolecule can be attached to the binding polymer. Once the biomolecule is attached to the binding polymer, the immobilized biomolecule is more available for binding. For example, immobilized proteins are less sterically hindered relative to when they are immobilized on supports when compared to supports that do not possess the ratio of reactive groups to ionizable groups as recited herein. Additionally, the disclosed binding polymers have greater flexibility, which also permit greater binding between the binding polymer and the biomolecule. The binding polymers can provide increased binding assay sensitivity and signal-to-noise ratios, which is a very desirable feature when conducting assays of biomolecules.

8. Preparation of dEMA Peptide-Modified Surface

The amount of binding polymer attached to the substrate can vary depending upon, for example, the selection of the binding polymer, the peptide, and the cell to be attached. In embodiments, the binding polymer can be, for example, at least one monolayer thick on the substrate surface. In embodiments, the binding polymer can have, for example, a thickness of about 10 A to about 2,000 A. In embodiments, the thickness of the binding polymer can have a lower endpoint of, for example, 10 Å, 20 Å, 40 Å, 60 Å, 80 Å, 100 Å, 150 Å, 200 Å, 300 Å, 400 Å, or 500 Å, including intermediate values and ranges, and an upper endpoint of 750 Å, 1,000 Å, 1,250 Å, 1,500 Å, 1,750 Å, or 2,000 Å, including intermediate values and ranges, where any lower endpoint can be combined with any upper endpoint to form the thickness range.

In embodiments, the binding polymer can be attached to or deposited on the substrate using techniques known in the art. For example, the substrate can be dipped in a solution of the binding polymer. In embodiments, the binding polymer can be sprayed, vapor deposited, screen printed, or robotically pin printed or stamped on the substrate. This could be done either on a fully assembled substrate or on a bottom insert (e.g., prior to attachment of the bottom insert to a holey plate to form a microplate).

In embodiments, the support can be made by attaching a binding polymer directly or indirectly to the substrate, wherein the binding polymer has a plurality of reactive groups capable of attaching to a biomolecule. When the binding polymer is directly or indirectly attached to the substrate, the binding polymer can be attached either covalently or non-covalently (e.g., electrostatic). FIG. 1 depicts one aspect of the attachment of the binding polymer of formula (III) to the substrate, where for example a nucleophilic surface group (Sur) (e.g., an amino group, hydroxyl group, or thiol group) reacts with an anhydride group of the binding polymer to produce a new covalent bond as represented by formula (II).

In embodiments, when the binding polymer is indirectly attached to the substrate, a tie layer can be used. The tie layer can be covalently or electrostatically attached to the outer surface of the substrate. The term “outer surface” with respect to the substrate is the region of the substrate that is exposed and can be subjected to manipulation. For example, any surface on the substrate that can come into contact with a solvent or reagent upon contact is considered the outer surface of the substrate. Thus, the tie layer can be attached to the substrate and the binding polymer. In embodiments, the substrates described herein can have a tie layer covalently bonded to the substrate; however, it is also contemplated that a different tie layer can be attached to the substrate by other means in combination with a tie layer that is covalently bonded to the substrate. For example, one tie layer can be covalently bonded to the substrate and a second tie layer can be electrostatically bonded to the substrate. In embodiments, when the tie layer is electrostatically attached to the substrate, the compound used to make the tie layer can be positively charged and the outer surface of the substrate can be treated such that a net negative charge exists so that tie layer compound and the outer surface of the substrate form an electrostatic bond.

In embodiments, the outer surface of the substrate can be derivatized so that there are groups capable of forming a covalent bond with the tie layer. The tie layer can be directly or indirectly covalently bonded to the substrate. In the situation where the tie layer is indirectly bonded to the substrate, a linker possessing group that can covalently attach to both the substrate and the tie layer can be used. Examples of linkers include, for example, an ether group, a polyether group, a polyamine, a polythioether and like groups, or combinations thereof. If a linker is not used, and the tie layer can be covalently bonded to the substrate, that is, directly covalently attached.

In embodiments, the tie layer can be derived from a compound comprising one or more reactive functional groups that can react with the binding polymer. The phrase “derived from” with respect to the tie layer means the resulting residue or fragment of the tie layer compound when it is attached to the substrate. The functional groups permit the attachment of the binding polymer to the tie layer. In embodiments, the functional groups of the tie layer compound can be, for example, an amino group, a thiol group, a hydroxyl group, a carboxyl group, an acrylic acid, an organic acid, an inorganic acid, an activated ester, an anhydride, an aldehyde, an epoxide, an isocyanate, an isothiocyanate, salts thereof, or a combination thereof.

In embodiments, the substrate can be amine-modified with, for example, a polymer comprising at least one amino group. Examples of such polymers include, but are not limited to, poly(lysine), poly(ethylenenimine), poly(allylamine), or silylated poly(ethylenenimine) or silylated poly(ethylenenimine). In embodiments, the substrate can be modified with an aminosilane. In embodiments, the tie layer can be derived from a straight or branched-chain aminosilane, aminoalkoxysilane, aminoalkylsilane, aminoarylsilane, aminoaryloxysilane, or like compounds, or salt thereof. In embodiments, the tie layer can be derived from 3-aminopropyl trimethoxysilane, N-(beta-aminoethyl)-3-aminopropyl trimethoxysilane, N-(beta-aminoethyl)-3-aminopropyl triethoxysilane, N′-(beta-aminoethyl)-3-aminopropyl methoxysilane, or aminopropylsilsesquixoxane.

In embodiments, when the substrate is comprised of gold, the binding polymer can be attached to the substrate by an aminothiol such as, for example, 11-amino-1-undecanethiol hydrochloride.

In embodiments, the tie layer can be attached to any of the described substrates using techniques known in the art. For example, the substrate can be dipped in a solution of the tie layer compound. In embodiments, the tie layer compound can be sprayed, vapor deposited, screen printed, or robotically pin printed or stamped on the substrate. This can be accomplished either on a fully assembled substrate or on a bottom insert (e.g., prior to attachment of the bottom insert to a holey plate to form a microplate).

In embodiments, the substrate can be a gold chip, the binding polymer can be a poly(ethylene-alt-maleic anhydride) indirectly attached to the substrate by an aminothiol, and the ratio of reactive groups to ionizable groups in the binding polymer can be from 0.67 to 3.0. In embodiments, the substrate can be a glass substrate with a layer comprising Ta₂O₅, Nb₂O₅, TiO₂, Al₂O₃, silicon nitride, SiO₂ or a mixture thereof, the binding polymer can be poly(ethylene-alt-maleic anhydride) indirectly attached to the substrate by a tie layer, wherein the tie layer is derived from aminopropylsilane (e.g., gamma-aminopropylsilane), and the ratio of reactive groups to ionizable groups in the binding polymer is from 0.67 to 3.0. In the above, the poly(ethylene-alt-maleic anhydride) can optionally be preblocked for obtain a polymer of the formula (III) with methoxyethyl amine, or like preblocking agents prior to attaching the polymer to the substrate.

Once the binding polymer has been attached to the substrate, one or more peptides can be attached to the binding polymer using the techniques presented above. In embodiments, when the biomolecule is a peptide, nucleic acid or protein, the nucleic acid or protein can be printed on the binding polymer using techniques known in the art. The amount of biomolecule that can be attached to the polymer layer can depend upon, for example, the biomolecule and binding polymer selected and the cell to be attached. In embodiments, one or more different biomolecules can be placed at different locations on the support. In the situation when different biomolecules are used, the biomolecules can be printed at the same time or different time.

In embodiments, the biomolecule can be deposited on (i.e., attached to) the support by immersing the tip of a pin into the composition comprising the biomolecule; removing the tip from the composition, wherein the tip comprises the composition; and transferring the composition to the support. This aspect can be accomplished, for example, by using a typographic pin array. The depositing step can be carried out using an automated, robotic printer. Such robotic systems are available commercially from, for example, Intelligent Automation Systems (IAS), Cambridge, Mass. The pin can be solid or hollow. The tips of solid pins are generally flat, and the diameter of the pins determines the volume of fluid that is transferred to the substrate. Solid pins having concave bottoms can also be used. In one aspect, to permit the printing of multiple arrays with a single sample loading, hollow pins that hold larger sample volumes than solid pins and therefore allow more than one array to be printed from a single loading can be used. Hollow pins include printing capillaries, tweezers and split pins. An example of a preferred split pen is a micro-spotting pin (TeleChem International, Sunnyvale, Calif.). In embodiments, pins made by Point Tech can be used. The spotting solutions described can be used in a number of commercial spotters including Genetix and Biorobotics spotters.

In embodiments, the peptide, or peptide combination can be deposited on the support in a pattern or combinatorial array or gradient. This can be useful for cell adhesion studies were one can desire to identify a new peptide sequence (in the example of an array) or peptide concentration (in the example of gradients) for a new anchorage dependent cell type. Cells can be incubated on the peptide modified surface containing a library of peptides sequences or a range of peptide concentrations to identify concentrations and sequences where cells adhere.

The peptide can be attached to the surface by methods known in the art. For example the peptide can be dissolved as a 1 mM concentration in borate buffer pH 9.2 and incubated with the anhydride surface. As an alternative, the anhydride surface can be hydrolyzed to the carboxylic acid form, followed by activation of the carboxylic acid group using known activation methods such as EDC/NHS activation, or like carbodiimide methods, and HATU, EEDQ, or like uronium/aminium methods. Such techniques have been reviewed in the literature (Hermanson, G. T., Bioconjugate Techniques, 2nd Ed.; Academic Press; Elsevier Inc., 2008). These types of conjugation methods can be particularly useful for a cell type that is sensitive to highly negative charged surfaces. Using the hydrolysis, activation, and blocking process, the amount of surface negative charge of the binding polymer can be minimized.

The peptide can be conjugated to the polymer at any suitable pH. In embodiments, the peptide can be conjugated at a pH between 7.4 and 9.2. For shorter amino acid sequences (e.g., 3-15 amino acids) a pH of 9.2 can be preferred. Not limited by theory, it is believed that the terminal amino groups are more reactive at pHs greater than 9. This can result in higher peptide densities for shorter amino sequences than if conjugated at a lower pH (e.g., pH 5) where the amine is less reactive towards the activated carboxyl.

In other instances where the anhydride surface can be completely hydrolyzed, the surface can be dehydrated using chemical means or elevated temperature under vacuum to regenerate the anhydride groups. This can be especially useful during the manufacturing of these types of surfaces where a long lag time can exist between the dEMA coating and peptide conjugation.

In embodiments, the disclosed binding polymer coated surface provides a surface to which any suitable adhesion peptide or combinations of peptides can be conjugated, providing an alternative to biological substrates or serum that have unknown components. In current cell culture practice, it is known that some cell types require the presence of a biological peptide or combination of peptides on the culture surface for the cells to adhere to the surface and be sustainably cultured. For example, HepG2/C3A hepatocyte cells can attach to plastic culture ware in the presence of serum. It is also known that serum can provide peptides that can adhere to plastic culture ware to provide a surface to which certain cells can attach. However, biologically-derived substrates and serum contain unknown components. For cells where the particular component or combination of components (peptides) of serum or biologically-derived substrates that cause cell attachment are known, those known peptides can be synthesized and applied to a dEMA surface as described herein to allow the cells to be cultured on a synthetic surface having no or very few components of unknown origin or composition.

For any of the disclosed peptides a conservative amino acid can be substituted for a specifically identified or known amino acid. A “conservative amino acid” refers to an amino acid that is functionally similar to a second amino acid. Such amino acids can be substituted for each other in a peptide with a minimal disturbance to the structure or function of the peptide according to well known techniques. The following five groups each contain amino acids that are conservative substitutions for others in the group: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); and Acidic: Aspartic acid (D), Glutamic acid (E), Asparagine (N), Glutamine (Q).

A linker or spacer, such as a repeating oligo- or poly(ethylene glycol) linker or any other suitable extending group, can be used to increase the distance between the peptide site and the polymer coated substrate. The linker can be of any suitable length. For example, if the linker is a repeating poly(ethylene glycol) linker, the linker can contain between 2 and 10 repeating ethylene glycol units. In embodiments, the linker can be a repeating poly(ethylene glycol) linker having about 4 repeating ethylene glycol units (see for example, commonly owned and assigned U.S. patent application Ser. No. 12/417,784, entitled “SURFACE FOR LABEL INDEPENDENT DETECTION AND METHOD THEREOF”). All, some, or none of the peptides can be conjugated to a maleic anhydride coated surface via linkers. Other potential linkers that can be used include, for example, peptide linkers such as poly(glycine) or poly(β-alanine). Any suitable conjugation techniques can be used to conjugate a linker to the peptide. In embodiments, amino acids themselves can serve as linkers or spacers. For example, additional amino acids can be inserted at the N- or C-terminus of a peptide to serve as a linker or spacer. In embodiments the linker includes polylysine, where the linker includes between 1 and 10 repeating lysine units; e.g. between 1 and 4 repeating lysine units.

A peptide can be conjugated to the binding polymer coated surface at any density, preferably at a density suitable to support culture of neuronal progenitor stem cells, primary human hepatocytes, undifferentiated stem cells, or other cell types. Peptides can be conjugated to a surface at a density of between about 1 μmol per mm² and about 50 μmol per mm² of surface. For example, the peptide can be present at a density of greater than 5, 6, 7, 8, 9, 10, 12, 15, or 20 μmol/mm² of the surface, including intermediate values and ranges. Standard BCA colorimetric techniques can be used to estimate peptide density. The amount of peptide present can vary depending on the composition of the binding polymer coating, the size and shape of the surface, and the nature of the peptide.

The level of peptide conjugated to the surface can be controlled in several ways. For example, different levels of peptide can be conjugated to the surface by varying the initial concentration of the peptide challenge solution that is reacted with the surface. Alternatively, the conjugation time can be adjusted to increase or decrease the amount of peptide conjugated. Furthermore, a species that competes with the peptide for reactive sites at the surface can be used to limit the amount of peptide bound to the surface.

The peptide may be cyclized or include a cyclic portion. Any suitable method for forming cyclic peptide can be employed. For example, an amide linkage can be created by cyclizing the free amino functionality on an appropriate amino-acid side chain and a free carboxyl group of an appropriate amino acid side chain. Also, a di-sulfide linkage can be created between free sulfhydryl groups of side chains appropriate amino acids in the peptide sequence. Any suitable technique can be employed to form cyclic peptides (or portions thereof). Methods described in, e.g., WO1989005150 can be employed to form cyclic peptides. Head-to-tail cyclic peptides, where the peptides have an amide bond between the carboxy terminus and the amino terminus can be employed. An alternative to the disulfide bond is, for example, a diselenide bond using two selenocysteines or mixed selenide/sulfide bond, e.g., as described in Koide, et al, 1993, Chem. Pharm. Bull., 41(3):502-6; Koide, et al., 1993, Chem. Pharm. Bull., 41(9):1596-1600; or Besse, et al., 1997, Journal of Peptide Science, vol. 3, 442-453.

Examples of peptides that can be conjugated to the dEMA modified surface includes KGGNGEPRGDTYRAY (SEQ ID NO:1), which is an RGD-containing sequence from bone sialoprotein with an additional “KGG” sequence added to the N-terminus. The lysine (K) serves as a suitable nucleophile for chemical conjugation, and the two glycine amino acids (GG) serve as spacers. Cystine (C), or another suitable amino acid, can alternatively be used for chemical conjugation, depending on the conjugation method employed. Of course, a conjugation or spacer sequence (KGG, KYG, or CGG, for example) can be present (e.g., to facilitate UV quantitation during stock and working solution preparation) or absent. Additional examples of suitable peptides for conjugation with maleic anhydride surfaces (with or without conjugation or spacer sequences) are peptides that include NGEPRGDTYRAY, (SEQ ID NO:2), GRGDSPK (SEQ ID NO:3) (short fibronectin) AVTGRGDSPASS (SEQ ID NO:4) (long FN), PQVTRGDVFTMP (SEQ ID NO:5) (Vitronectin), RNIAEIIKDI (SEQ ID NO:6) (lamininβ1), KYGRKRLQVQLSIRT (SEQ ID NO:7) (mLMα1 res 2719-2730), NGEPRGDTRAY (SEQ ID NO:8) (BSP-Y), NGEPRGDTYRAY (SEQ ID NO:9) (BSP), KYGAASIKVAVSADR (SEQ ID NO:10) (mLMα1 res2122-2132), KYGKAFDITYVRLKF (SEQ ID NO:11) (mLMγ1 res 139-150), KYGSETTVKYIFRLHE (SEQ ID NO:12) (mLMγ1 res 615-627), KYGTDIRVTLNRLNTF (SEQ ID NO:13) (mLMγ1 res 245-257), TSIKIRGTYSER (SEQ ID NO:14) (mLMγ1 res 650-261), TWYKIAFQRNRK (SEQ ID NO:15) (mLMα1 res 2370-2381), SINNNRWHSIYITRFGNMGS (SEQ ID NO:16) (mLMα1 res 2179-2198), KYGLALERKDHSG (SEQ ID NO:17) (tsp1 RES 87-96), or GQKClVQTTSWSQCSKS (SEQ ID NO:18) (Cyr61 res 224-240).

In embodiments, the peptide comprises KGGK⁴DGEPRGDTYRATD¹⁷ (SEQ ID NO:19), where Lys⁴ and Asp¹⁷ together form an amide bond to cyclize a portion of the peptide; KGGL⁴EPRGDTYRD¹³ (SEQ ID NO:20), where Lys⁴ and Asp¹³ together form an amide bond to cyclize a portion of the peptide; KGGC⁴NGEPRGDTYRATC¹⁷ (SEQ ID NO:21), where Cys⁴ and Cys'⁷ together form a disulfide bond to cyclize a portion of the peptide; KGGC⁴EPRGDTYRC¹³ (SEQ ID NO:22), where Cys⁴ and Cys¹³ together form a disulfide bond to cyclize a portion of the peptide, or KGGAVTGDGNSPASS (SEQ ID NO:23).

In embodiments, the peptide can be acetylated, amidated, or both. Any peptide or peptide sequence can be conjugated to a disclosed surface.

In embodiments, the peptide polymer surface composition can contain multiple peptide sequences. These sequences can be directed toward the adhesion of either a single cell type or to enable multiple cell types to adhere to the same surface.

9. Cell Culture on dEMA Synthetic Peptide-Modified Surfaces

A cell culture article containing a dEMA synthetic peptide modified surface and culture media can be seeded with cells. The surface employed can be selected based on the type of cell being cultured. The cells can be of any cell type. For example, the cells can be connective tissue cells, epithelial cells, endothelial cells, hepatocytes, skeletal or smooth muscle cells, heart muscle cells, intestinal cells, kidney cells, or cells from other organs, stem cells, islet cells, blood vessel cells, lymphocytes, cancer cells, primary cells, cell lines, or the like. The cells can be mammalian cells, preferably human cells, but can also be non-mammalian cells such as bacterial, yeast, or plant cells.

In embodiments, the cells can be stem cells which refer to cells that have the ability to continuously divide (self-renewal) and that are capable of differentiating into a diverse range of specialized cells. In embodiments, the stem cells are multipotent, totipotent, or pluripotent stem cells that can be isolated from an organ or tissue of a subject. Such cells are capable of giving rise to a fully differentiated or mature cell types. A stem cell can be, for example, a bone marrow-derived stem cell, autologous or otherwise, a neuronal stem cell, or an embryonic stem cell. A stem cell can be nestin positive. A stem cell can be, for example, a hematopoietic stem cell. A stem cell can be, for example, a multi-lineage cell derived from epithelial and adipose tissues, umbilical cord blood, liver, brain or other organ. In embodiments, the stem cells can be, for example, pluripotent stem cells, such as pluripotent embryonic stem cells isolated from a mammal. Suitable mammals can include, for example, rodents such as mice or rats, primates including human and non-human primates. In embodiments, the maleic anhydride surface with a conjugated peptide supports undifferentiated culture of embryonic stem cells for 5 or more passages, 7 or more passages, or 10 or more passages, including intermediate values and ranges. Typically stems cells are passaged to a new surface after they reach about 75% confluency. The time for cells to reach 75% confluency is dependent on media, seeding density, and other like factors.

Because human embryonic stem cells (hESC) have the ability to grown continually in culture in an undifferentiated state, the hESC for use with synthetic peptide surfaces as described herein can be obtained from an established cell line. Examples of human embryonic stem cell lines that have been established include, for example, H1, H7, H9, H13 or H14 (available from WiCell, U. Wisconsin) (Thompson (1998) Science, 282:1145); hESBGN-01, hESBGN-02, hESBGN-03 (BresaGen, Inc., Athens, Ga.); HES-1, HES-2, HES-3, HES-4, HES-5, HES-6 (from ES Cell International, Inc., Singapore); HSF-1, HSF-6 (from U. California, San Francisco); I 3, I 3.2, I 3.3, I 4, I 6, I 6.2, J 3, J 3.2 (derived at the Technion-Israel Institute of Technology, Haifa, Israel); UCSF-1 and UCSF-2 (Genbacev, et al., Fertil. Steril., 83(5):1517-29, 2005); lines HUES 1-17 (Cowan et al., NEJM, 350(13):1353-56, 2004); and line ACT-14 (Klimanskaya, et al., Lancet, 365(9471):1636-41, 2005). Embryonic stem cells can also be obtained directly from primary embryonic tissue. Typically this is done using frozen in vitro fertilized eggs at the blastocyst stage, which would otherwise be discarded.

Other sources of pluripotent stem cells include induced primate pluripotent stem (iPS) cells. iPS cells refer to cells, obtained from a juvenile or adult mammal, such as a human, that are genetically modified, e.g., by transfection with one or more appropriate vectors, such that they are reprogrammed to attain the phenotype of a pluripotent stem cell such as an hESC. Phenotypic traits attained by these reprogrammed cells include morphology resembling stem cells isolated from a blastocyst as well as surface antigen expression, gene expression and telomerase activity resembling blastocyst derived embryonic stem cells. The iPS cells typically have the ability to differentiate into at least one cell type from each of the primary germ layers: ectoderm, endoderm and mesoderm. The iPS cells, like hESC, also form teratomas when injected into immuno-deficient mice, e.g., SCID mice (see Takahashi, et al., (2007) Cell, 131(5):861; Yu, et al., (2007) Science, 318:5858).

To maintain stem cells in an undifferentiated state it can be desirable to minimize non-specific interaction or attachment of the cells with the surface, while obtaining selective attachment to the peptide(s) attached to the surface. The ability of stem cells to attach to the surface without conjugated peptide can be tested prior to conjugating peptide to determine whether the synthetic peptide surface provides for little to no non-specific interaction or attachment of stem cells. Once a suitable surface has been selected, cells can be seeded in culture medium on the surface.

Prior to seeding cells, the cells, regardless or cell type, can be harvested and suspended in a suitable medium, such as a growth medium in which the cells are to be cultured once seeded. For example, the cells can be suspended in and cultured in a serum-containing medium, a conditioned medium, or a chemically-defined medium. “Chemically-defined medium” means cell culture media that contains no components of unknown composition. Chemically defined cell culture media can, in various embodiments, contains no proteins, hydrozylates, or peptides of unknown composition. In embodiments, chemically defined media contains peptides or proteins of known composition, such as recombinant growth hormones. Because all components of chemically-defined media have a known chemical structure, variability in culture conditions and thus variability in cell response can be reduced, increasing reproducibility. In addition, the possibility of contamination is reduced. Further, the ability to scale up is made easier due, at least in part, to the factors discussed above. Chemically defined cell culture media are commercially available from Invitrogen (Carlsbad, Calif.) as STEMPRO®, a fully serum- and feeder-free (SFM) specially formulated from the growth and expansion of embryonic stem cells, Xvivo (Lonza), and Stem Cell Technologies, Inc. as mTeSR™ 1 maintenance media for human embryonic stem cells.

One or more growth or other factors can be added to the medium in which cells are incubated with the surface modified with peptide. The factors can facilitate cellular proliferation, adhesion, self-renewal, differentiation, or like aspects. Examples of factors that can be added to or included in the medium include muscle morphogenic factor (MMP), vascular endothelium growth factor (VEGF), interleukins, nerve growth factor (NGF), erythropoietin, platelet derived growth factor (PDGF), epidermal growth factor (EGF), activin A (ACT) such as activin A, hematopoietic growth factors, retinoic acid (RA), interferons, fibroblastic growth factors, such as basic fibroblast growth factor (bFGF), bone morphogenetic protein (BMP), peptide growth factors, heparin binding growth factor (HBGF), hepatocyte growth factor, tumor necrosis factors, insulin-like growth factors (IGF) I and II, transforming growth factors, such as transforming growth factor-β1 (TGFβ1), and colony stimulating factors.

The cells can be seeded at any suitable concentration. Typically, the cells are seeded at about 10,000 cells/cm² of surface to about 500,000 cells/cm². For example, cells can be seeded at about 50,000 cells/cm² of substrate to about 150,000 cells/cm². However, higher and lower concentrations can readily be used. The incubation time and conditions, such as temperature, CO₂ and O₂ levels, growth medium, and like considerations, will depend on the nature of the cells being cultured and can be readily modified. The time that the cells are cultured with the surface can vary depending on the cell response desired.

The cultured cells can be used for any suitable purpose, including, for example: obtaining sufficient amounts of undifferentiated stem cells cultured on a synthetic surface in a chemically defined medium for use in investigational studies or for developing therapeutic uses; for investigational studies of the cells in culture; for developing therapeutic uses; for therapeutic purposes; for studying gene expression, e.g., by creating cDNA libraries; for studying drug and toxicity screening; and the like purposes.

One suitable way to determine whether cells are undifferentiated is to determine the presence of the OCT4 marker. In embodiments, the undifferentiated stems cells cultured on synthetic peptide surfaces as described herein for 5, 7, or 10 or more passages retain the ability to be differentiated.

The neural progenitor stem cells and human embryonic stem cells can be cultured to about 80% confluence in chemically defined serum-free media on flat surfaces and can be maintained in an undifferentiated state for at least one passage.

Other particularly useful aspects and considerations of the disclosed process and materials, include for example:

The dEMA peptide-modified surfaces (e.g., BSP, VN and FN peptides) that were prepared and characterized, overcome many limitations of animal derived laminin protein materials.

Biospecific specific attachment of the cells to the modified polymer surface via peptide linkage in known media (e.g., serum-free culture) was accomplished.

Unlike the freshly coated laminin surface, the dEMA-peptide-modified surfaces were stable and did not need to be freshly prepared just prior to each use. This provides off-the-shelf ease of use and convenience.

The dEMA-peptide-modified surfaces are amenable to current dEMA manufacturing processes, for example, as used for EPIC® well-plate manufacture for biochemical assay and cell culture.

The dEMA peptide-modified surfaces are relatively low cost, and provide a scalable manufacturing process with no activation step necessary.

The peptides are relatively inexpensive compared to a whole protein.

The synthetic surfaces address and solve issues such as shelf-life.

The dEMA peptide-modified surfaces provide a replacement for laminin coated Epic® surfaces. The replacement surfaces have been shown to support growth and differentiation of neural progenitor cells.

The dEMA peptide-modified surfaces can be formed on surfaces other than glass, such as polystyrene or TOPAS®.

The collagen peptide-modified dEMA surfaces overcome limitations of animal derived Matrigel® and Collagen, such as minimizing lot-to-lot variability.

Biospecific attachment of cells via surface immobilized peptides are amenable to serum-free culture of primary hepatocytes.

Unlike collagen and Matrigel™ coated substrates, the dEMA-collagen peptide-modified surfaces are stable and do not require specific storage conditions (cf., collagen is stored at 4° C. and Matrigel™ at −20° C.). This provides off-the-shelf ease of use and convenience.

A coating of dEMA with a collagen peptide attached is thin (10 to 200 nm, 50 to 100 nm) enough to perform label-independent Epic® platform hepatocyte assays, overcoming some of the limitations of the collagen coating for EPIC® assays such as reproducibility and manufacturability.

Collagen peptide-modified dEMA surfaces can be extended to other cell lines.

In embodiments, the disclosure provides a method for making a synthetic peptide cell culture article, comprising:

attaching a pre-blocked polymer directly or indirectly attached to a substrate, the pre-blocked polymer has a plurality of anhydride reactive groups capable of attaching to a biomolecule and a plurality of carboxy groups, the ratio of reactive groups to carboxy groups can be, for example, from 0.5 to 10.0; and

contacting the pre-blocked polymer attached to a substrate and a peptide, to for the peptide modified pre-blocked polymer surface.

In embodiments, the pre-blocked polymer can be, for example, the product of the a maleic anhydride containing polymer and a pre-block agent or source is selected from water, ammonia, 2-(2-aminoethoxy)ethanol, N,N-dimethyl ethylenediamine, ethanolamine, ethylenediamine, hydroxylamine, methoxyethyl amine, ethyl amine, isopropyl amine, butyl amine, propyl amine, hexyl amine, 2-amino-2-methyl-1-propanol, 2-(2-aminoethyl amino) ethanol, 2-(2-aminoethoxy)ethanol, dimethylethanolamine, dibutyl ethanolamine, 1-amino-2-propanol, polyethylene glycol, polypropylene glycol, 4,7,10-trioxa-1,13-tridecanediamine, polyethylene glycol or an amine-terminated-polyethylene glycol, Trizma hydrochloride, or any combination thereof.

In embodiments, the pre-blocked polymer can be prepared, for example, by combining a blocking agent and a maleic anhydride copolymer attached to the surface.

In embodiments, the disclosure provides a cell culture article comprising:

a substrate having a polymer of the formula (I) directly or indirectly attached to a surface of the substrate:

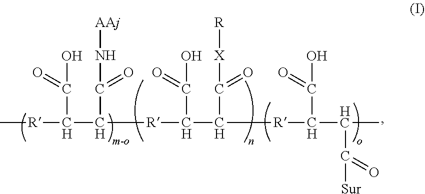

where

m-o is an integer representing the mers containing a carboxy group and an AAⱼ peptide-modified group,

n is an integer representing the mers containing a pre-blocked group (X—R) and a carboxy group,

o is an integer representing the mers containing a carboxy group and surface attachment group,

AAⱼ comprises at least one covalently attached peptide comprised of an AAⱼ peptide-modification source having amino acids,

j is an integer representing from 5 to 50 amino acids,

Sur comprises a surface attachment group,

X is a divalent —NH—, —O—, or —S— of a pre-block source,

R is H, or a substituted or an unsubstituted, linear or branched, alkyl group, an oligo(ethylene oxide), an oligo(ethylene glycol), or a dialkyl amine of the pre-block source,

R′ is a substituted or an unsubstituted, linear or branched, hydrocarbylene having from 2 to about 10 carbon atoms,

the relative mer ratio (m-o:n:o) is from about 0.5:1:0.01 to about 10:1:0.001, and salts thereof.

The AAⱼ can be sourced or derived from, for example, at least one of Ac-KGGPQVTRGDVFTMP-NH₂, GRGDSPK-NH₂, Ac-KGGAVTGRGDSPASS-NH₂, and Ac-KGGNGEPRGDTYRAY, or a combination thereof.

The pre-block agent or pre-block source can be, for example, an alkyl amine, an alkylhydroxy amine, an alkoxyalkyl amine, an alcohol, an alkyl thiol, water, or H₂S. One example of a pre-block agent or pre-block source can be methoxyethyl amine, i.e., in the polymer, X is NH, and R is —CH₂—CH₂—OCH₃.

The substrate can be, for example, a plastic, a polymeric or co-polymeric substance, a ceramic, a glass, a metal, a crystalline material, a noble or semi-noble metal, a metallic or non-metallic oxide, an inorganic oxide, an inorganic nitride, a transition metal, or any combination thereof. The substrate can be, for example, a glass with a layer comprising Ta₂O₅, Nb₂O₅, TiO₂, Al₂O₃, SiO₂, silicon nitride, or a mixture thereof, and the layer is optionally adjacent to the surface of the glass. The substrate can be, for example, at least one of a microplate, an array, a slide, a container, a vessel, a microcarrier bead, a dish, a flask, or a combination thereof. The well-plate or array can include, for example, at least 24, 96, 384, 1536, or like distinct and defined locations.

The polymer of formula (I) can be, for example, indirectly attached to the substrate by a tie layer, the tie layer being covalently attached to the outer surface of the substrate. The polymer can be, for example, attached to the substrate. The substrate can be modified with, for example, an aminosilane or a polymer comprising at least one amino group. Alternatively or additionally, the substrate can be modified with, for example, poly-lysine, poly(ethyleneimine), poly(allylamine), silylated poly(ethyleneimine), and like polymeric amines, or combinations thereof. Alternatively or additionally, the tie layer can be, for example, electrostatically attached to the outer surface of the substrate. The ratio of peptide containing groups to pre-block containing groups (m-o:n) can be, for example, from 0.5 to 5.0. The ratio of peptide groups to pre-blocked groups (m-o:n) can be, for example, from 0.67 to 3.0, including intermediate values and ranges.

The carboxy group can be, for example, at least one of: a positively charged group, a negatively charged group, a zwitter ion group, or a combination thereof. In embodiments the positively charged group can be, for example, an ammonium group and the negatively charged group comprises a carboxylate, a sulfonate, a phosphonate group, or a combination thereof.

In embodiments, the disclosure provides a method for cell culture comprising:

contacting the cell culture article as described above with cells, wherein the peptide-modified, pre-blocked polymer surface attracts and retains the cells. The cells can be, for example, selected from neural progenitor cells, neural stem cells, neurons, glial cells, astrocytes, neuronal cell lines (PC12), embryonic stem cells, iPS cells, other stem cells, fibroblast (3T3, MRCS), hepatocyte cell lines (HUH7, HepG2, HepG2/C3A, Fa2N-4), and primary mammalian hepatocytes.

In embodiments, the disclosure provides a method of making the cell culture article as described above comprising:

contacting a pre-blocked polymer of the formula (III):

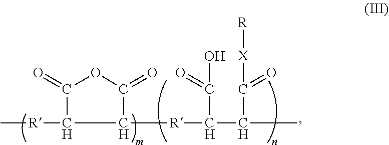

where

—X—R is a pre-block source residue,

X is a divalent —NH—, —NR—, —O— or —S—;

R is H, or a substituted or an unsubstituted, linear or branched, alkyl group, an oligo(ethylene oxide), an oligo(ethylene glycol), or a dialkyl amine;

R′ is a residue of a first unsaturated monomer that has been copolymerized with maleic anhydride;

the relative ratio (m:n) of the maleic anhydride reactive groups (m) to the pre-blocked groups (n) is from 0.5 to 10,

with a silane-modified surface to form a pre-blocked polymer-modified surface of the formula (II):

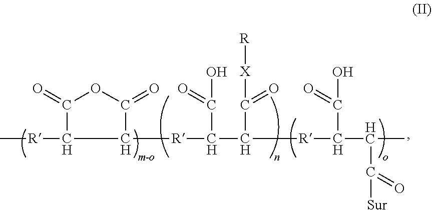

where

Sur is a divalent surface attachment group; and

contacting the pre-blocked polymer-modified surface of the formula (II) with a peptide of the formula: H₂N-AAⱼ, to form the pre-blocked peptide-modified polymer surface of the formula (I):

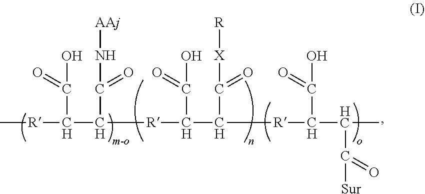

where

AAj represents a covalently bonded peptide, and j is an integer from 5 to 50, and salts thereof.

In embodiments, the disclosure provides a method for making a cell culture article, comprising

reacting at least one peptide with substantially all of the maleic anhydride reactive groups of a pre-blocked polymer attached to a surface to form a pre-blocked peptide-modified polymer surface.

The polymer can be, for example, poly(ethylene-alt-maleic anhydride) and the peptide-modifier (AA). The peptide-modification source can be, for example, a sequence selected from Ac-KGGNGEPRGDTYRAY-NH₂ (BSP), Ac-KGGPQVTRGDVFTMP-NH₂ (VN), and like modifications, or a combination thereof.

These peptide-modified surfaces showed superior proliferation and differentiation of neural progenitor cells cultured under serum-free conditions.

Referring to the Figures, FIG. 1 schematically shows the process used to attach peptide sequences to dEMA surfaces. dEMA surfaces were freshly prepared on well plate inserts using a published APS/dEMA preparative process (see commonly owned and assigned U.S. Pat. No. 7,781,203; specific matter incorporated by reference includes Examples 1, and 6 to 13) and assembled into 96-well format, or directly obtained as 96-well plates. The synthetic peptides were conjugated directly to the dEMA surfaces via the epsilon-amine group of the lysine residue in pH 9 borate buffer at concentrations ranging from 1.9 microM to 0.5 milliM for 30 minutes. After conjugation, the surfaces were blocked with ethanolamine.

FIG. 2 shows microscopic images of neural progenitor cells cultured on dEMA surface coated peptides Ac-KGGNGEPRGDTYRAY-NH₂ (BSP) (FIG. 2A); GRGDSPK (short FN) (FIG. 2B); Ac-KGGAVTGRGDSPASS-NH₂ (long FN) (FIG. 2C) and Ac-KGGPQVTRGDVFTMP-NH₂ (VN) (FIG. 2D).

FIG. 3 shows progenitor stem cells on the peptide surfaces of FIGS. 3A to 3D were differentiated for six days by the removal of growth factors. All four peptide-modified surfaces supported the differentiation process.

FIG. 4 shows growth undifferentiated (FIG. 4A) and growth differentiated (FIG. 4B) neural progenitor stem cells on freshly coated laminin (an industry standard for these cells).

FIG. 5 shows day 6 immuno-staining of differentiated neural progenitor cells on dEMA surfaces modified with the collagen-peptide sequences 10 (Ac-KGGCKRARGDDMDDYC-NH₂) and 11 (Ac-KGGGRGDTP-NH₂) from Table 2 and a freshly coated laminin surface. Cells were grown for three days in undifferentiated state in the presence of growth factors. The surfaces conjugated with peptides 7 (Ac-KGGGFRGDGQ-NH₂), 10 (Ac-KGGCKRARGDDMDDYC-NH₂), and 11 (Ac-KGGGRGDTP-NH₂) from Table 2 supported undifferentiated cell growth for three days. Differentiation protocol, which comprises of culturing in the absence of growth factors, was followed for six days. The surfaces prepared with peptide 10 (Ac-KGGCKRARGDDMDDYC-NH₂), supported the differentiation of cells in clusters with entangled neuron processes clustered in regions. While the surfaces prepared with peptide 11 (Ac-KGGGRGDTP-NH₂) supported differentiation of neural cells to neurons (identified by β-tubulin III marker in FIG. 5) comparable to freshly prepared laminin, and promoted increase differentiation of neural progenitor cells to astrocytes (identified by GFAP marker in FIG. 5) relative to laminin.

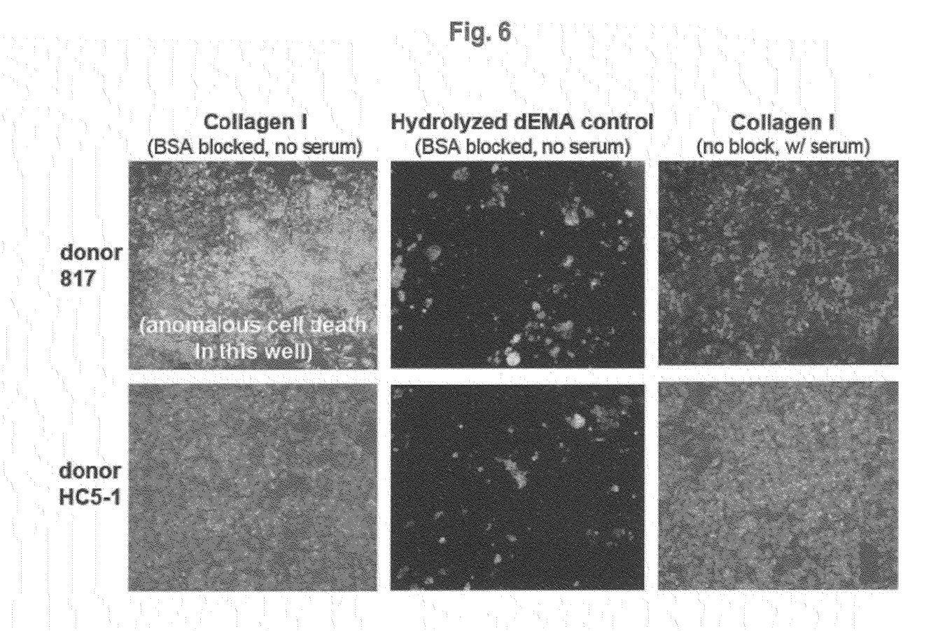

FIG. 6 shows images of cell viability of primary hepatocytes from donors 817 and HC5-1 cultured on Collagen I (with and without serum) and hydrolyzed dEMA (no peptide attached) control surfaces (green=live, red=dead, at 5× magnification) evaluated by Live/Dead staining at day seven of culture. When cultured with or without serum, Collagen I surfaces supported attachment, spreading, and long term retention of hepatocytes from donors 817 and HC5-1 with no noticeable loss of cells. On the hydrolyzed dEMA control surface (without peptides), cells from both donors attached and formed aggregates within a 48 hour period, but detached and were gradually lost from the surface with daily media changes.

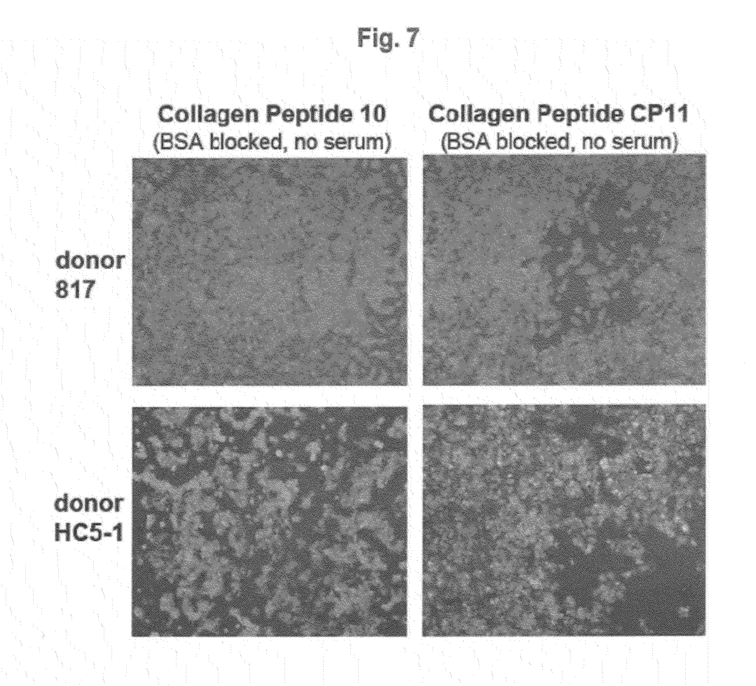

FIG. 7 shows images of cell viability of primary hepatocytes from donors 817 and HC5-1 cultured serum-free on dEMA surfaces conjugated with collagen peptides 10 (Ac-KGGCKRARGDDMDDYC-NH₂) and 11 (Ac-KGGGRGDTP-NH₂) (green=live, red=dead, at 5× magnification) evaluated by Live/Dead staining at day seven of culture. When cultured without serum, many cells from both donors attached within a 48 hr period and remained attached up to day seven of culture. These surfaces supported attachment, spreading, and long term retention similar to Collagen I control surfaces from FIG. 6 with no noticeable loss of cells.

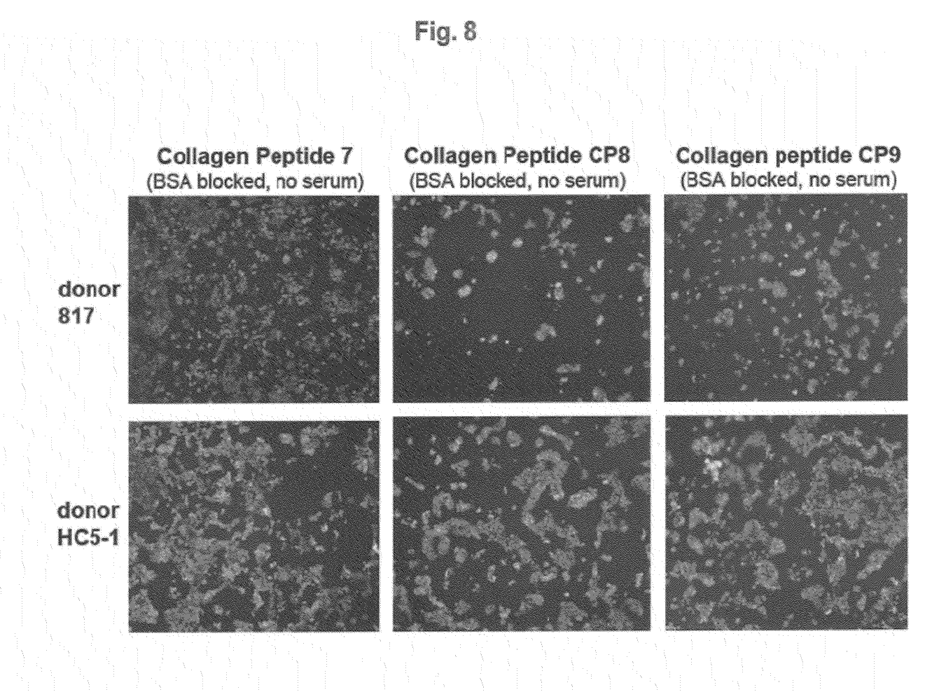

FIG. 8 shows day seven images of cell viability of primary hepatocytes from donors 817 and HC5-1 cultured serum-free on dEMA surfaces conjugated with collagen peptides 7 (Ac-KGGGFRGDGQ-NH₂), 8 (Ac-KGGCGGFHRRIKA-NH₂), and 9 (Ac-KGGGWKTSRTSHTC-NH₂) (green=live, red=dead, at 5× magnification) evaluated by Live/Dead staining at day seven of culture. When cultured without serum, many cells from both donors attached and formed aggregates within a 48 hr period, but some cells detached between days four to seven with daily media changes.

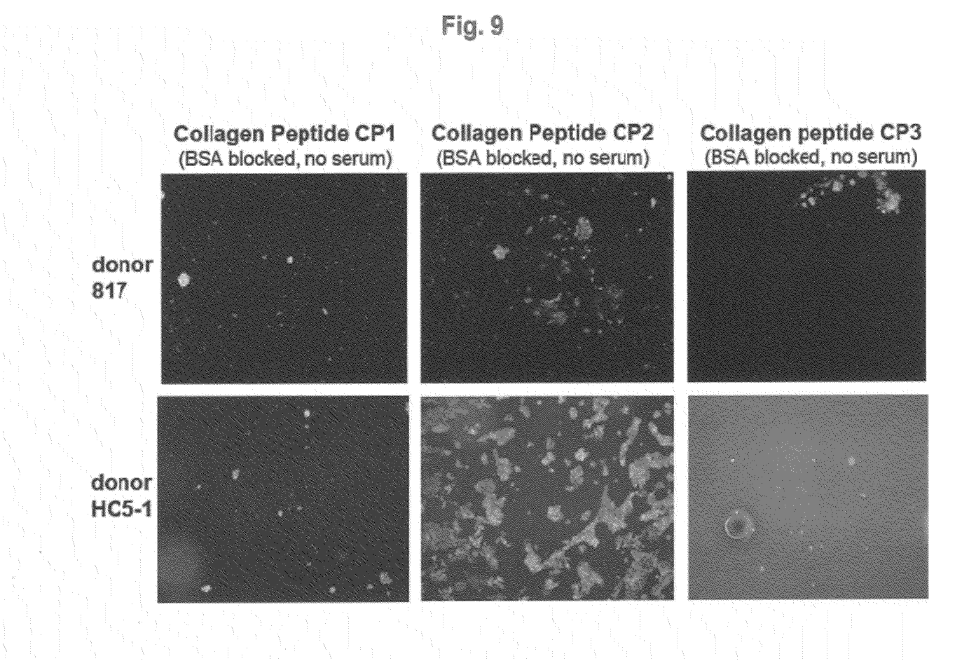

FIG. 9 shows day seven images of cell viability of primary hepatocytes from donors 817 and HC5-1 cultured serum-free on dEMA surfaces conjugated with collagen peptides 1 (Ac-KGGCGGDGEAG-NH₂), 2 (Ac-KGGCWKTSLTSHTC-NH₂) and 3 (Ac-KGGGASGERGPO-NH₂) (green=live, red=dead, at 5× magnification) evaluated by Live/Dead staining at day seven of culture. When cultured without serum, cells from both donors cells attached and formed aggregates within a 48 hour period, but gradually detached between days four to seven with daily media changes (similar to dEMA control surface without the conjugated collagen peptide). Peptides 4 (Ac-KGGGLOGERGRO-NH₂), 5 (Ac-KGGGFOGERGVQ-NH₂), 6 (Ac-TAGSCLRKFSTMGGK-NH₂), and 12 (Ac-KGGGPOGFOGERGPO-NH₂) gave similar results.

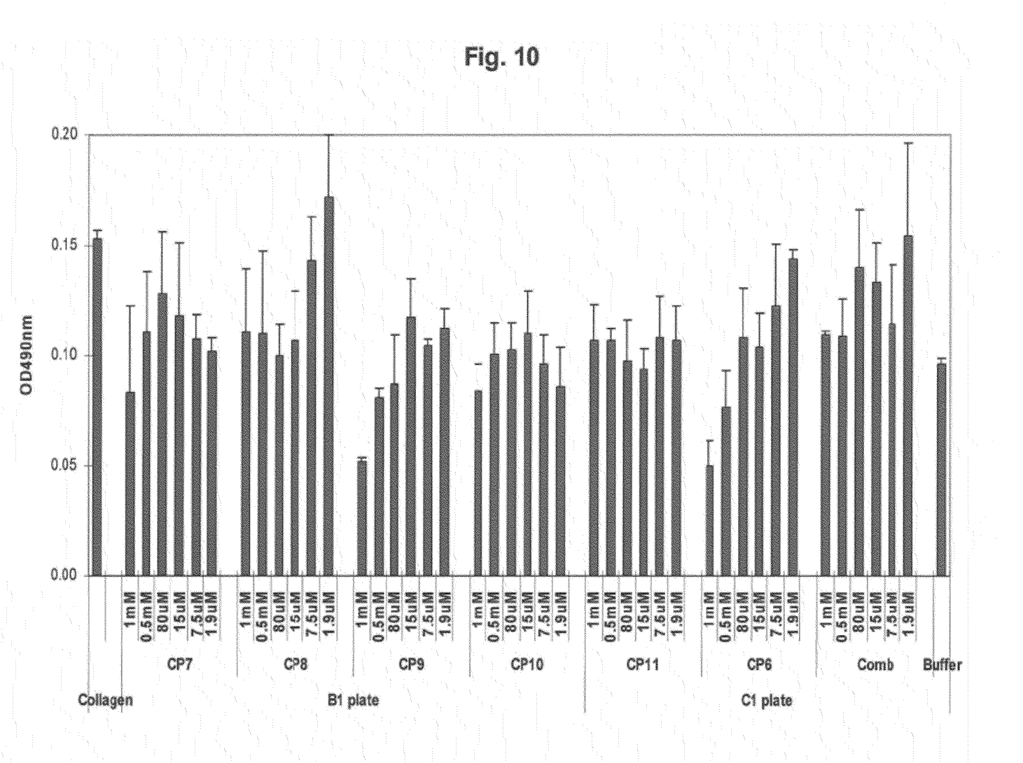

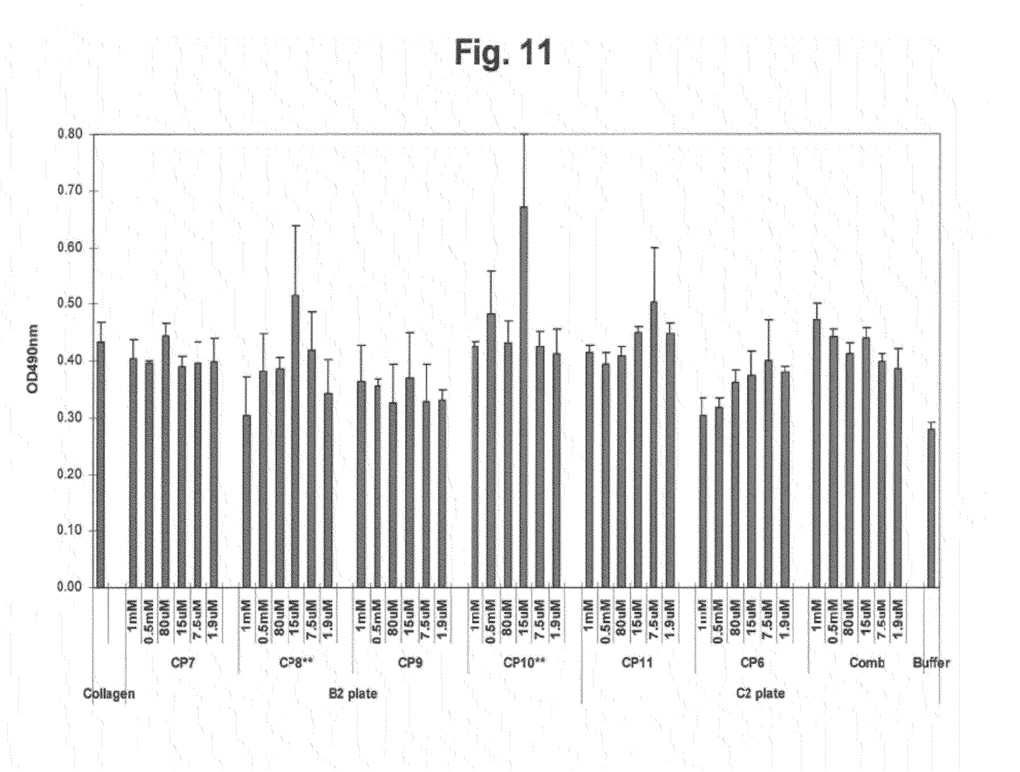

FIG. 10 shows 24 hr cell number data of primary hepatocytes from donor 817 in serum-free media cultured on dEMA surfaces conjugated with collagen peptides (evaluated by MTS quantitative assay). In general, collagen peptide conjugated dEMA surfaces supported good cell attachment that was similar to Collagen I. For each surface, various concentrations of the peptides were conjugated to dEMA. The data indicates that the surfaces support good cell attachment even at a lower concentration of peptides, for example, 1.9 micromolar to 0.5 millimolar, with the exception of CP9 and CP6. FIG. 11 shows day seven data for cell number of primary hepatocytes from donor 817 in serum-free media cultured on dEMA surfaces conjugated with collagen peptides (evaluated by MTS quantitative assay). Collagen peptide conjugated dEMA surfaces support cell attachment and seven days cell retention equal to Collagen I. For each surface various concentrations of the peptides were conjugated to dEMA. The data indicate that only a very low concentration, for example, 1.9 micromolar to 0.5 millimolar of the peptide is sufficient to obtain a surface that supports good cell retention for at least 1 week of culture with daily media change.

### EXAMPLES

The following examples serve to more fully describe the manner of using the above-described disclosure, and to further set forth the best modes contemplated for carrying out various aspects of the disclosure. It is understood that these examples do not limit the scope of this disclosure, but rather are presented for illustrative purposes. The working examples further describe how to the methods and make the articles of the disclosure.

### Example 1

General procedure for peptide conjugation on a maleic anhydride copolymer (dEMA) coated surface in a 96-well format. All peptides (derived from collagen or laminin) were dissolved at 1 micromolar in 100 micromolar borate buffer solution, pH 9.2. In some instances the peptide solution was serially diluted to a lower concentration, for example, down to 1.9 micromolar. In a 96-well plate layout (8×12), 50 microliters of each peptide solution was introduced into each available well (except for the indicated negative control wells containing only buffer) and conjugated for 30 minutes. The peptide solution was aspirated, and then 50 microliters of 1 molar ethanolamine pH 8 was added to each well for 15 minutes followed by aspiration. The wells were then washed with phosphate buffered saline, pH 7.4 (PBS, 3×100 microliters), 1% sodium dodecyl sulfate (SDS), (1×100 microliters for 15 minutes on an orbital shaker), DI water (5×100 microliters), and ethanol (2×100 microliters) and air dried about 16 hours at 25° C. For negative control surfaces, the lower wells of the dEMA 96-well plate were treated with pH 9 borate buffer only (without peptide) and processed similarly to peptide surfaces.

### Example 2

General procedure for neural stem cell culture on synthetic collagen and laminin peptide-modified surfaces. Plates with peptides were washed with 70% ethanol and dried (evaporated) for about 16 to 20 hrs in a laminar flow hood. Next, the plates were washed twice with 1×PBS. A solution bovine serum albumin (BSA, 1% in PBS, 50 microliters) was added to each well containing the peptides and the control wells, and then incubated for 5 hrs at 37° C. As a positive control, row G in columns 1-6, were coated with laminin (50 microliters, 20 micrograms/milliliter) and also incubated with the BSA blocked wells. The wells were then washed with 1×PBS and the cells were seeded at 20,000 cells per well. For undifferentiated cells, the cultures were grown for 9 days. Media was changed every day with growth factor supplementation. For differentiated cells, the growth factor deprived media was added after 3 days of seeding and cells were grown further for 6 days with media changes every other day.

Phase contrast microscopy. After the experiment, the cells were fixed with 4% paraformaldhyde and washed three times with 1×PBS. Cells were assessed using a Ziess Axiovert 200M inverted Brightfield/fluorescence microscope. Undifferentiated cells were confluent and compact in shape without any processes as shown in FIGS. 2A-D, and 4A, while differentiated cells showed distinct processes protruding from the cells as shown in FIGS. 3A-D, and 4B.

### Example 3

General procedure for immunostaining of neural stem cells. After the intended growth period, cells were fixed with 4% paraformaldehyde for 20 min followed by three washes with 1×PBS. Blocking solution containing 1×PBS and 5% donkey serum and Triton X-100 was added and incubated at about 25° C. for 2 hrs. Primary antibodies for B-tubulin III, GFAP with appropriate dilutions were added to the cells and incubated for about 16-20 hrs at 4° C. Next, the cells were washed with 1×PBS three times and incubated with Cy3 and FITC secondary antibodies for 1 hr at about 25° C. After washing with 1×PBS twice, the staining of the cells was assessed using a Ziess Axiovert 200M inverted Brightfield/fluorescence microscope using Cy3 and FITC channels (FIG. 5). Nuclei were stained with Hoechst.

### Example 4

General procedure for primary hepatocyte cell culture on synthetic collagen peptide-modified surfaces. All surfaces were blocked with 1% BSA to ensure that the interactions were specific to the conjugated peptides. Human primary hepatocytes were seeded (plated) on the surface at 60,000 cells in 100 microliters of serum-free plating media (in house media preparation, similar to media commercially available from XenoTech) per well in 96 well microplate format and cultured at 37° C., in a humidified atmosphere of 5% CO₂. Cells were maintained in serum-free MFE maintenance medium (in house media preparation, similar to media commercially available from XenoTech) with daily medium change and microscopically observed daily to monitor cell culture health and morphology.

Quantification of primary hepatocyte adhesion and retention. The number of cells in culture (24 hour cell attachment and 7 day retention) was quantified using CellTiter96® Aqueous One Solution MTS assay (Promega #G3581) and enclosed (standard) protocol after washing away cells that are not adhered to the surface, see FIGS. 10 and 11.

Assessment of primary hepatocytes viability. Live/Dead® Viability/Cytotoxicity Kit *for mammalian cells* (Cat #: L-3229) and the enclosed (standard) protocol was used to assess cell viability after one week in culture and cultures were imaged using a Ziess Axiovert 200M inverted Brightfield/fluorescence microscope using FITC channel (see FIGS. 6 to 9).

The disclosure has been described with reference to various specific embodiments and techniques. However, it should be understood that many variations and modifications are possible while remaining within the scope of the disclosure.

1. A cell culture article comprising:
a substrate having a polymer of the formula (I) directly or indirectly attached to a surface of the substrate: 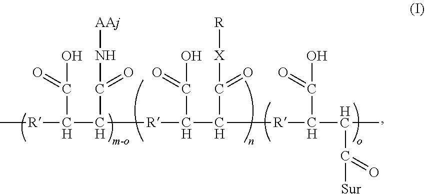
where
m-o is an integer representing the mers containing a carboxy group and an AAⱼ peptide-modified group,
n is an integer representing the mers containing a pre-blocked group (X—R) and a carboxy group,
o is an integer representing the mers containing a carboxy group and surface attachment group,
AAⱼ comprises at least one covalently attached peptide comprised of an AAⱼ peptide-modification source having amino acids,
j is an integer representing from 5 to 50 amino acids,
Sur comprises a surface attachment group,
X is a divalent —NH—, —O—, or —S— of a pre-block source,
R is H, or a substituted or an unsubstituted, linear or branched, alkyl group, an oligo(ethylene oxide), an oligo(ethylene glycol), or a dialkyl amine of the pre-block source,
R′ is a substituted or an unsubstituted, linear or branched, hydrocarbylene having from 2 to about 10 carbon atoms,
the relative mer ratio (m-o:n:o) is from about 0.5:1:0.01 to about 10:1:0.001, and salts thereof.

2. The cell culture article of claim 1 wherein AAⱼ comprises at least one of:
(SEQ ID NO: 10)
Ac-KGGPQVTRGDVFTMP-NH₂,
(SEQ ID NO: 3)
GRGDSPK,
(SEQ ID NO: 4
Ac-KGGAVTGRGDSPASS-NH₂,
and
(SEQ ID NO: 5)
Ac-KGGNGEPRGDTYRAY-NH₂,
or a combination thereof.

3. The cell culture article of claim 1 wherein the pre-block agent or source comprises an alkyl amine, an alkylhydroxy amine, an alkoxyalkyl amine, an alcohol, an alkyl thiol, water, or H₂S.

4. The cell culture article of claim 1 wherein the pre-block agent or source comprises methoxyethyl amine.

5. The cell culture article of claim 1, wherein the substrate comprises a plastic, a polymeric or co-polymeric substance, a ceramic, a glass, a metal, a crystalline material, a noble or semi-noble metal, a metallic or non-metallic oxide, an inorganic oxide, an inorganic nitride, a transition metal, or any combination thereof.

6. The cell culture article of claim 1, wherein the substrate is at least one of a microplate, an array, a slide, a container, a vessel, a microcarrier bead, a dish, a flask, or a combination thereof.

7. The cell culture article of claim 1, wherein the polymer of formula (I) is indirectly attached to the substrate by a tie layer, the tie layer being covalently attached to the outer surface of the substrate.

8. The cell culture article of claim 1, wherein the ratio of peptide containing groups to pre-block containing groups (m-o:n) is from 0.5 to 5.0.

9. The cell culture article of claim 1, wherein the carboxy group comprises at least one of: a positively charged group, a negatively charged group, a zwitter ion group, or a combination thereof.

10. The cell culture article of claim 9, wherein the positively charged group comprises an ammonium group and the negatively charged group comprises a carboxylate, a sulfonate, a phosphonate group, or a combination thereof.

11. The cell culture article of claim 1, wherein the AAⱼ peptide-modification source is of the formula:
(SEQ ID NO: 1)
(XₐX_{b}) PQVTRGDVFTMP
(X_{c}X_{d}),
(SEQ ID NO: 1)
(XₐX_{b})PQVTRGDVFTMP,
or
(SEQ ID NO: 1)
PQVTRGDVFTMP
(X_{c}X_{d}),
where
Xₐ and X_{d} are primary amine containing moieties, and
X_{b} and X_{c} are optional hydrophilic linker moieties.

12. The method of making of claim 1 wherein the peptide-modification source is a sequence selected from:
(SEQ ID NO: 5)
Ac-KGGNGEPRGDTYRAY-NH₂
(BSP),
(SEQ ID NO: 10)
Ac-KGGPQVTRGDVFTMP-NH₂
(VN),
and a combination thereof.

13. A method for cell culture comprising:
contacting the cell culture article of claim 1 with cells, wherein the peptide-modified, pre-blocked polymer surface attracts and retains the cells.

14. The method of claim 13 wherein the cells are selected from neural progenitor cells, neural stem cells, neurons, glial cells, astrocytes, neuronal cell lines (PC12), embryonic stem cells, iPS cells, other stem cells, fibroblast (3T3, MRCS), hepatocyte cell lines, primary mammalian hepatocytes, and combinations thereof.

15. A method of making the cell culture article of claim 1 comprising:
contacting a pre-blocked polymer of the formula (III): 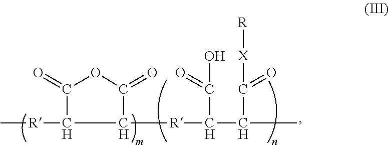
where
X—R is a pre-block source residue,
X is a divalent —NH—, —NR—, —O—, or —S—;
R is H, or a substituted or an unsubstituted, linear or branched, alkyl group, an oligo(ethylene oxide), an oligo(ethylene glycol), or a diallyl amine;
R′ is a residue of a first unsaturated monomer that has been copolymerized with maleic anhydride; the relative ratio (m:n) of the maleic anhydride reactive groups (m) to the pre-blocked groups (n) is from 0.5 to 10,
with a silane-modified surface to form a pre-blocked polymer-modified surface of the formula (II): 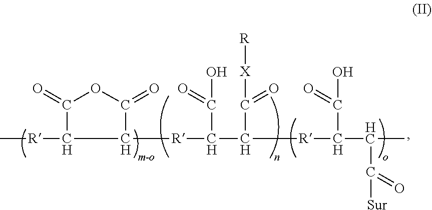
where
Sur is a divalent surface attachment group; and
contacting the pre-blocked polymer-modified surface of the formula (II) with a peptide of the formula: H₂N-AAⱼ, to form the pre-blocked peptide-modified polymer surface of the formula (I): 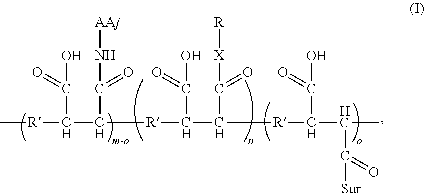
where
AAj represents a covalently bonded peptide, and j is an integer from 5 to 50, and salts thereof.

16. A method for making a cell culture article, comprising
reacting at least one peptide source with substantially all of the maleic anhydride reactive groups of a pre-blocked polymer attached to a surface to form a pre-blocked peptide-modified polymer surface.

17. The method of making of claim 16, wherein the polymer comprises poly(ethylene-alt-maleic anhydride) and the peptide source is of the formula:
(SEQ ID NO: 1)
(XₐX_{b})PQVTRGDVFTMP
(X_{c}X_{d}),
(SEQ ID NO: 1)
(XₐX_{b})PQVTRGDVFTMP,
or
(SEQ ID NO: 1)
PQVTRGDVFTMP
(X_{c}X_{d}),
where
Xₐ and X_{d} are primary amine containing moieties, and
X_{b} and X_{c} are optional hydrophilic linker moieties.

18. The method of making of claim 16, wherein the peptide source is a sequence selected from:
(SEQ ID NO: 5)
Ac-KGGNGEPRGDTYRAY-NH₂
(BSP),
(SEQ ID NO: 10)
Ac-KGGPQVTRGDVFTMP-NH₂
(VN),
and a combination thereof.
